SEQUENCE LISTING (2 sequences)

SEQ ID NO: 1 - protein, 496 aa, Homo Sapiens
MAAPEPARAAPPPPPPPPPPPGADRVVKAVPFPPTHRLTSEEVFDLDGIPRVDVLKNHLVKEGRVDEEIALRIINEGAAILRREKTMIEVEAPITVCGDIHGQFFDLMKLFEVGGSPANTRYLFLGDYVDRGYFSIECVLYLWVLKILYPSTLFLLRGNHECRHLTEYFTFKQECKIKYSERVYEACMEAFDSLPLAALLNQQFLCVHGGLSPEIHTLDDIRRLDRFKEPPAFGPMCDLLWSDPSEDFGNEKSQEHFSHNTVRGCSYFYNYPAVCEFLQNNNLLSIIRAHEAQDAGYRMYRKSQTTGFPSLITIFSAPNYLDVYNNKAAVLKYENNVMNIRQFNCSPHPYWLPNFMDVFTWSLPFVGEKVTEMLVNVLSICSDDELMTEGEDQFDGSAAARKEIIRNKIRAIGKMARVFSVLREESESVLTLKGLTPTGMLPSGVLAGGRQTLQSGNDVMQLAVPQMDWGTPHSFANNSHNACREFLLFFSSCLSS

SEQ ID NO: 2 - DNA, 1800 bp, Homo Sapiens
gggccctacagagggtccgrcatgctccccggcggcgccgccgcttggctctggtagccgccgcccccgcccccaaccccgcccggcccagagcctagccgagccccgggcccagcatggccgccccggagccggcccgggctgcaccgcccccacccccgcccccgccgcccccccccggggctgaccgcgtcgtcaaagctgtccctttccccccaacacatcgcttgacatctgaagaagtatttgatttggatgggatacccagggttgatgttctgaagaaccacttggcgaaagaaggtcgagtagatgaagaaattgcgcttagaattatcaatgagggtgctgccatccttcggagagagaaaaccatgatagaagtagaagctccaatcacagtgtgtggtgacatccatggccaattttttgatctgatgaaactttttgaagtaggaggatcacctgctaatacacgatacctttttcttggcgattatgtggacagaggttattttagtatagagtgtgtcttatatttatgggttctgaagattctatacccaagcacattatttcttctgagaggcaaccatgaatgcagacaccttactgaatattttacctttaagcaggaatgtaaaattaagtattcggaaagagtctatgaagcttgtatggaagcttttgatagtttgcctcttgctgcacttttaaaccaacagtttctttgtgttcatggtggactttcaccagaaatacacacactggatgatattaggagattagatagattcaaagagccacctgcatttggaccaatgtgtgacttgttatggtccgatccttctgaagattttggaaatgaaaaatcacaggaacattttagtcacaatacagttcgaggatgttcttatttttataactatccagcagtgtgtgaatttttgcaaaacaataatttgttatcgattattagagctcatgaagctcaagatgcaggctatagaatgtacagaaaaagtcaaactacagggttcccttcattaataacaattttttcggcacctaattacttagatgtctacaataataaagctgctgtattaaagtatgaaaataatgtgatgaatattcgacagtttaactgttctccacatccttactggttgcctaattttatggatgtcttcacgtggtctttaccgtttgttggagaaaaagtgacagaaatgttggtaaatgttctgagtatttgctctgatgatgaactaatgactgaaggtgaagaccagtttgatggttcagctgcagcccggaaagaaatcataagaaacaaaattcgagcaattggcaagatggcaagagtcttctctgttctcagggaggagagtgaaagtgtgctgacactcaagggcctgactcccacagggatgttgcccagtggagtgttagctggaggacggcagaccctgcaaagtggtaatgatgttatgcaacttgctgtgcctcagatggactggggcacacctcactcttttgctaacaattcacataatgcatgcagggaattccttctgttttttagttcctgtctcagcagctgacctagacagggtagtgtattagctagtgtctcattaatacgtgatcagggcagaaaactgatagaatgggtattcctttcaattgaaaataatggtcagttcctcagcttttcatgaaatgatatgggagcagctcatatcataatgtctgaaatatttatttattcatctgtctaattcacccttttctttt

(19) United States
(12) Patent Application Publication
(10) Pub. No.: US 2009/0047259 A1
(43) Pub. Date: Feb. 19, 2009

(54) Methods of Using the Calcineurin A Variant CnA-beta 1

(76) Inventors: Enrique Lara, Madrid (ES); Nadia Rosenthal, Rome (IT)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/158,509

(22) PCT Filed: Dec. 18, 2006
(86) PCT No.: PCT/EP2006/012202

§ 371 Date: Sep. 22, 2008

Related U.S. Application Data
(60) Provisional application No. 60/753,277, filed on Dec. 22, 2005.

(51) Int. Cl. A61K 48/00; A61K 31/7088; C12Q 1/68; A61K 31/7105; C12Q 1/02

(52) U.S. Cl. 424/93.7; 514/44; 435/29; 435/6

(57) ABSTRACT

The present invention relates to an inhibitor or activator of the calcineurin subunit Aβ1 isoform (CnAβ1) and the production of a medicament for the treatment of skeletal muscle injury or degeneration or cancer in a subject. CnAβ1 is constitutively active, cyclosporin-insensitive, highly expressed in proliferating myoblasts and human tumors, where it inhibits FoxO transcription factors independently of its phosphatase activity. The CnAβ1 isoform is a candidate for interventional strategies in muscle wasting, and a target for cancer treatment.

The present invention relates to an inhibitor or activator of the calcineurin subunit Aβ1 isoform (CnAβ1) and the production of a medicament for the treatment of skeletal muscle injury or degeneration or cancer in a subject. CnAβ1 is constitutively active, cyclosporin-insensitive, highly expressed in proliferating myoblasts and human tumors, where it inhibits FoxO transcription factors independently of its phosphatase activity. The CnAβ1 isoform is a candidate for interventional strategies in muscle wasting, and a target for cancer treatment. All references cited herein are hereby incorporated herein by reference.

The calcium/calmodulin dependent serine/threonine phosphatase calcineurin plays a central role in skeletal and cardiac muscle hypertrophy and regeneration. In response to calcium increase, calcineurin induces dephosphorylation, nuclear translocation and activation of NFAT, a process sensitive to the action of the immunosuppressive drug Cyclosporin-A.

Calcineurin (Cn) consists of a catalytic (CnA) and a regulatory (CnB) subunit. CnA isoforms are composed of a catalytic domain, a CnB-interacting domain, a calmodulin binding region and an autoinhibitory domain, which maintains the enzyme inactive in the absence of calcium raise. Three CnA genes have been described: CnAα and CnAβ are ubiquitously expressed, whereas CnAγ is expressed in brain and testis. A naturally occurring CnAβ splicing variant lacking the autoinhibitory domain was described (termed CnAβ1, as opposed to the predominant CnAβ2 isoform), although its function remained obscure (Guerini D, Klee C B. Cloning of human calcineurin A: evidence for two isozymes and identification of a polyproline structural domain. Proc Natl Acad Sci USA. 1989 December; 86(23):9183-7.).

In skeletal muscle, the Cn/NFAT pathway mediates initial myotube differentiation, enhances myoblast recruitment, controls muscle fiber type specification and ameliorates injury to dystrophic muscles (B. B. Friday, V. Horsley, G. K. Pavlath, J. Cell Biol. 149, 657 (2000); V. Horsley et al., J. Cell Biol. 153, 771 (2002), V. Horsley, K. M. Jansen, S. T. Mills, G. K. Pavlath, Cell 113, 483 (2003); F. J. Naya et al., J. Biol. Chem. 275, 4545 (2000); S. A. Parsons, B. J. Wilkins, O. F. Bueno, J. D. Molkentin, Mol. Cell. Biol. 23, 4331 (2003); N. Stupka et al., J. Physiol. 575, 645 (2006). The function of the CnAβ1 isoform has not been explored, since no obvious phenotype was reported in a germline knockout of the CnAβ gene (O. F. Bueno, E. B. Brandt, M. E. Rothenberg, J. D. Molkentin, Proc. Natl. Acad. Sci. USA 99, 9398 (2002)). Notably, in that study the knockout scheme involved deletion of catalytic domain encoded by exon 2, which would still allow transcription of in-frame transcripts encoding phosphatase-dead CnAβ2 or CnAβ1 protein. Increased CnAβ1 expression was noted in response to the muscle-restricted overexpression of a local IGF-1 isoform (mIGF-1), which preserved muscle integrity and enhanced satellite cell activity in a mouse model of amyotrophic lateral sclerosis (ALS) (G. Dobrowolny et al., J. Cell Biol. 168, 193 (2005)).

Calcineurin has been involved in treatments aiming to counteract muscle wasting and atrophy. In the murine model (mdx) for the Duchenne Muscular Dystrophy, treatment with the glucocorticoid deflazacort, activated calcineurin and induced NFAT-dependent genes, including the dystrophin homologue utrophin, and restored myocyte viability.

U.S. Pat. No. 6,362,160 describes immunophilin-binding agents that inhibit the phosphatase calcineurin, leading to the increased phosphorylation of certain brain proteins, including nitric oxide synthase. Immunophilin-binding drugs can be used therapeutically in the treatment of vascular stroke and neurodegenerative disorders such as Alzheimer's disease and Huntington's disease.

U.S. Pat. No. 7,084,241 describes specific inhibitors of NFAT activation by calcineurin and their use in treating immune-related diseases. Methods for high throughput screening of candidate agents are described. Pharmaceutical compositions are also provided. It is stated that immunosuppressive agents shall be identified which selectively inhibit the calcineurin-NFAT interactions and which do not inhibit the enzymatic activity of calcineurin for its other substrates.

U.S. Pat. No. 6,875,581 describes a method for screening of modulators of calcineurin is provided, which uses the interaction between calcineurin and superoxide dismutase. Modulators of calcineurin are potential candidates for drugs, e.g. for immunosuppressive drugs.

U.S. Pat. No. 7,041,437 describes FoxO3a as a therapeutic and diagnostic tool for impaired glucose tolerance conditions.

In summary, CnAβ1 has been identified as a crucial target to provide and develop new substances suitable as drugs, especially as drugs that can be used for an effective treatment of skeletal muscle injury or skeletal muscle degeneration, or cancer in a subject. In view of this, it is clear that there is a need in the art for agents that inhibit CnAβ1. These agents could provide an advance in the treatment of skeletal muscle injury or skeletal muscle degeneration, or cancer.

This object of the invention, in a first aspect thereof, is solved by the use of an inhibitor or activator of the calcineurin subunit API isoform (CnAβ1) having the amino acid sequence according to SEQ ID No. 2 for the production of a medicament for the treatment of skeletal muscle injury or degeneration or cancer in a subject.

Transgenic over-expression of CnAβ1 in post-mitotic skeletal muscle enhances muscle regeneration, reduces fibrosis and induces a faster resolution of inflammation. In contrast, mice over-expressing a full version of CnAα containing the autoinhibitory domain, present increased fibrosis and inflammation and delayed muscle regeneration. In uninjured subjects, CnAβ1 induces a fast-to-slow fiber switch, accompanied by IKKα activation and extensive NFATc dephosphorylation. Unlike artificially truncated Cn isoforms, which also lack the autoinhibitory domain, CnAβ1 constitutively activates NFATc in a cyclosporine A-insensitive manner and does not require the conventional Cn-dephosphorylating sites in NFATc. Thus, CnAβ1 represents a unique physiological form of calcineurin with new physiological and molecular properties that provide increased tissue regenerative capacity.

The inventors describe for the first time that CnAβ1, a CnA naturally occurring form, enhances skeletal muscle regeneration by decreasing the presence of macrophages, the expression of the TGF-P1 and the production of extracellular matrix. Regenerating muscles from CnAα transgenic mice however show increased number of macrophages and fibrosis and delayed muscle regeneration. The inventors could also show that CnAβ1 expression is transiently induced after skeletal muscle injury.

In addition, CnAβ1 was found capable of constitutively activating NFAT and inducing a fast-to-slow muscle fiber switch. These data offer a new perspective of the regulation and function of calcineurin, broadening the understanding of this intriguing molecule and opening new roads for the development of calcineurin-based therapeutic strategies.

Preferred is a use according to the present invention, wherein the inhibitor or activator of CnAβ1 is selected from an inhibitor or activator of the biological activity of CnAβ1 and/or an inhibitor or activator of the expression of CnAβ1.

In general, the present invention provides for inhibitors or activators (modulators) of CnAβ1 that either affect the biological activity of CnAβ1 and/or the expression of CnAβ1 in a cell, tissue or organ. With “the biological activity of CnAβ1” is meant that the inhibitor or activator modulates the enzymatic activities of the CnAβ1, such as, for example, activities of the catalytic domain, the calmodulin-binding region or the C-terminal domain. A modulator of the “expression of CnAβ1” means an inhibitor or activator that modifies the amount of CnAβ1, the amount of CnAβ1-encoding mRNA, and/or the posttranslational modification of CnAβ1 (if any). The modulator can either directly (such as an antisense oligonucleotide) or indirectly (such as modulators that act on regulatory elements or the splicing machinery) affect the expression of CnAβ1. Modulators of the invention can be identified, for example, through screening methods as described herein below.

Thus, another aspect of the present invention relates to an inhibitor or activator of the expression of CnAβ1 that is selected from a vector encoding calcineurin CnAβ1, antisense oligonucleotides (such as RNA, DNA, PNA and other nucleic acid derivatives, and combinations thereof), siRNA and miRNA, and respective uses thereof. Preferably, said vector is a viral vector, which allows for an efficient gene transfer. In certain embodiments, the therapeutically effective amount of the inhibitor or activator is administered by providing to the subject a nucleic acid encoding the inhibitor or activator, and expressing the peptide fragment or biologically active analog thereof in vivo.

Another aspect of the present invention relates to an inhibitor or activator of the biological activity of CnAβ1 that is selected from a vector encoding biologically inactive CnAβ1, biologically inactive CnAβ1, an inhibitory peptide, a phosphatase inhibitor, and an inhibitor of the activity of a transcription factor of the FoxO family (see also below), and respective uses thereof.

Yet another aspect of the present invention relates to an inhibitor or activator of CnAβ1 that comprises cells that recombinantly express CnAβ1, and respective uses thereof.

Preferred is a use according to the present invention, wherein the subject to be treated is a mammal, preferably a human (i.e. a human patient).

Preferred is a use according to the present invention, wherein the treatment of skeletal muscle injury or degeneration or cancer comprises preventing or reversing skeletal or cardiac muscle atrophy, muscle wasting, enhancing skeletal muscle regeneration, decreasing scar formation in injured skeletal muscle, inducing myoblast differentiation, and blocking tumor cell growth.

Treating is meant to include, e.g., preventing, treating, reducing the symptoms of, or curing the disease or condition.

The invention also includes a method for treating a subject at risk for a disease as above, wherein a therapeutically effective amount of an inhibitor or activator is provided. Being at risk for the disease can result from, e.g., a family history of the disease, a genotype which predisposes to the disease, or phenotypic symptoms which predispose to the disease.

Another aspect of the present invention relates to a use as above, wherein the medicament further comprises additional pharmaceutically active ingredients that modulate calcineurin, such as glucocorticoids or cyclosporin A. Nevertheless, since CnAβ1 is capable of activating NFAT without increasing the intracellular calcium concentration and this activation is insensitive to the immuno-suppressor cyclosporine A (CsA), CnAβ1 induction/delivery could be used in those cases where NFAT activation is needed but CsA is being used as a treatment. For instance, myoblast transplant can be used for the treatment of muscle injury or degenerative diseases. However, an immuno-suppressant is needed in order to prevent the host-versus-graft reaction. When CsA is administered, rejection is prevented but regeneration is impaired. Expression of CnAβ1 in the transplanted myoblasts could overcome this impairment but would still allow inhibition of the host immune response by CsA.

The invention also includes a method for screening a compound for the ability to inhibit or activate CnAβ1, comprising contacting a cell with a compound suspected to inhibit or activate CnAβ1; assaying the contents of the cell to determine the amount and/or biological activity of CnAβ1; and comparing the determined amount and/or biological activity of CnAβ1 to a predetermined level, wherein a change of said amount and/or biological activity of CnAβ1 is indicative for a compound that inhibits or activates CnAβ1. Preferred is a method according to the invention, wherein the cell is a skeletal muscle cell. Further preferred is a method according to the invention, wherein the amount of calcineurin Aβ1 mRNA is determined. Other applications of the inventors' findings include a method for the screening of drugs capable of selectively inducing or inhibiting CnAβ1 and/or other CnA isoforms. In one preferred embodiment, screening is done by quantitative real-time RT-PCR using specific primers for each isoform.

Another aspect of the present invention then relates to a method for producing a pharmaceutical composition, comprising a method for screening according to the present invention, and formulating the screened compound with pharmaceutically acceptable carriers and/or excipients. Another aspect of the present invention then relates to a pharmaceutical composition, produced as above. Preferred is the composition according to the invention, wherein said composition is an antisense composition or a composition comprising cells that recombinantly express CnAβ1.

In certain embodiments of the invention, the administration can be designed so as to result in sequential exposures to the compound over some time period, e.g., hours, days, weeks, months or years. This can be accomplished by repeated administrations of the compound, e.g., by one of the methods described above, or alternatively, by a controlled release delivery system in which the compound is delivered to the subject over a prolonged period without repeated administrations. By a controlled release delivery system is meant that total release of the compound does not occur immediately upon administration, but rather is delayed for some time. Release can occur in bursts or it can occur gradually and continuously. Administration of such a system can be, e.g., by long acting oral dosage forms, bolus injections, transdermal patches or subcutaneous implants. Examples of systems in which release occurs in bursts include, e.g., systems in which the compound is entrapped in liposomes which are encapsulated in a polymer matrix, the liposomes being sensitive to a specific stimulus, e.g., temperature, pH, light, magnetic field, or a degrading enzyme, and systems in which the compound is encapsulated by an ionically-coated microcapsule with a microcapsule core-degrading enzyme. Examples of systems in which release of the compound is gradual and continuous include, e.g., erosional systems in which the compound is contained in a form within a matrix, and diffusional systems in which the compound permeates at a controlled rate, e.g., through a polymer. Such sustained release systems can be, e.g., in the form of pellets or capsules.

The compound can be administered prior to or subsequent to the appearance of disease symptoms. In certain embodiments, the compound is administered to patients with familial histories of the disease, or who have phenotypes that may indicate a predisposition to the disease, or who have been diagnosed as having a genotype which predisposes the patient to the disease, or who have other risk factors.

The compound is administered to the subject in a therapeutically effective amount. By therapeutically effective amount is meant that amount which is capable of at least partially preventing or reversing the disease. A therapeutically effective amount can be determined on an individual basis and will be based, at least in part, on consideration of the species of the subject, the subject's size, the subject's age, the efficacy of the particular compound used, the longevity of the particular compound used, the type of delivery system used, the time of administration relative to the onset of disease symptoms, and whether a single, multiple, or controlled release dose regimen is employed. A therapeutically effective amount can be determined by one of ordinary skill in the art employing such factors and using no more than routine experimentation.

In certain preferred embodiments, the concentration of the compound if it is a peptide is at a dose of about 0.1 to about 1000 mg/kg body weight/day, more preferably at about 0.1 to about 500 mg/kg/day, more preferably yet at about 0.1 to about 100 mg/kg/day, and most preferably at about 0.1 to about 5 mg/kg/day. Preferably, the dosage form is such that it does not substantially deleteriously affect the subject.

Another aspect of the present invention then relates to a method of modulating the expression of calcineurin Aβ1 in skeletal muscle tissue of a subject comprising administering a composition as above to the subject such that the expression of calcineurin Aβ1 in the skeletal muscle tissue of the subject is modulated.

Another aspect of the present invention then relates to a method of enhancing skeletal muscle regeneration in a subject comprising expressing or overexpressing calcineurin Aβ1 in or proximate to skeletal, in particular injured, muscle tissue in the subject.

Yet another aspect of the present invention then relates to a method of treating skeletal muscle injury in a subject comprising expressing or overexpressing calcineurin Aβ1 in or proximate to skeletal, in particular injured, muscle tissue in the subject. Still another aspect of the present invention then relates to a method of treating skeletal muscle degeneration in a subject comprising expressing or overexpressing calcineurin Aβ1 in or proximate to degenerated or degenerating skeletal muscle tissue in the subject.

Another aspect of the present invention then relates to a method of treating cancer in a subject, comprising administering to said subject an effective amount of an inhibitor of the expression and/or biological activity of CnAβ1, as described herein.

Another aspect of the present invention then relates to a method of inducing cellular differentiation by inhibiting the expression of CnAβ1 in myoblasts and/or tumor cells.

Preferred is a method according to the invention as above, wherein expressing or overexpressing calcineurin Aβ1 comprises delivering cells that express calcineurin Aβ1 to said subject.

CnAβ1 enhances skeletal muscle regeneration and decreases scar formation after injury. Thus, in one embodiment of the present invention, the inventors propose to express CnAβ1 in the muscle in order to treat skeletal muscle injuries and degeneration. CnAβ1 expression in the muscle can be achieved, for example, by any method known to those of skill in the art, including (1) the use of a composition capable of selectively inducing the alternative splicing of the CnAβ mRNA, giving rise to CnAβ1, or (2) delivering appropriate vectors (e.g., viral vectors) to the muscle, thus resulting in CnAβ1. A third possibility would be to deliver cells to the injured muscle that would be engineered to express CnAβ1. These cells would have a paracrine effect on the damaged muscle and would enhance regeneration.

Other aspects of the present invention then relate to a method for blocking tumor cell growth by inhibiting CnAβ1 expression, a method for preventing or reversing skeletal and cardiac muscle atrophy by overexpressing CnAβ1, a method of inhibiting the activity of the transcription factors of the FoxO family by overexpressing CnAβ1, and a method of activating the transcription factors of the FoxO family by inhibiting CnAβ1 expression using iRNA. How to overexpress or to inhibit CnAβ1 in these instances, is known to the person of skill.

Specifically, according to the invention, CnAβ1 is regarded as an effective target in order to provide a method for blocking tumor cell growth, since a) CnAβ1 is strongly induced in tumour tissue; b) CnAβ1 knockdown reduces tumour cell proliferation; and c) CnAβ1 knockdown induces the expression of tumour suppressor genes.

In certain embodiments, the therapeutically effective amount of the peptide fragment is provided by providing to the subject a composition comprising subject cells wherein a recombinant nucleic acid having a nucleotide sequence encoding the peptide fragment has been introduced ex vivo into the subject cells so as to express the peptide fragment in the subject cells. The peptide fragment is administered to the subject by administering the subject cells having the recombinant nucleic acid. Preferably, the recombinant nucleic acid is a gene therapy vector, e.g., as described herein. Preferably, the subject cells are derived from the subject to be treated or allogeneic cells.

In certain embodiments, the therapeutically effective amount of the peptide fragment is provided by providing to the subject a composition comprising subject cells wherein a recombinant nucleic acid having a nucleotide sequence encoding the peptide fragment has been introduced ex vivo into the subject cells so as to express the peptide fragment in the subject cells. The peptide fragment is administered to the subject by administering the subject cells having the recombinant nucleic acid. Preferably, the recombinant nucleic acid is a gene therapy vector, e.g., as described herein. Preferably, the subject cells are derived from the subject to be treated or allogeneic cells.

Administration of an agent, e.g., a peptide or nucleic acid can be accomplished by any method which allows the agent to reach the target cells. These methods include, e.g., injection, deposition, implantation, suppositories, oral ingestion, inhalation, topical administration, or any other method of administration where access to the target cells by the agent is obtained. Injections can be, e.g., intravenous, intradermal, subcutaneous, intramuscular or intraperitoneal. Implantation includes inserting implantable drug delivery systems, e.g., microspheres, hydrogels, polymeric reservoirs, cholesterol matrices, polymeric systems, e.g., matrix erosion and/or diffusion systems and non-polymeric systems, e.g., compressed, fused or partially fused pellets. Suppositories include glycerin suppositories. Oral ingestion doses can be enterically coated. Inhalation includes administering the agent with an aerosol in an inhalator, either alone or attached to a carrier that can be absorbed. The agent can be suspended in liquid, e.g., in dissolved or colloidal form. The liquid can be a solvent, partial solvent or non-solvent. In many cases, water or an organic liquid can be used.

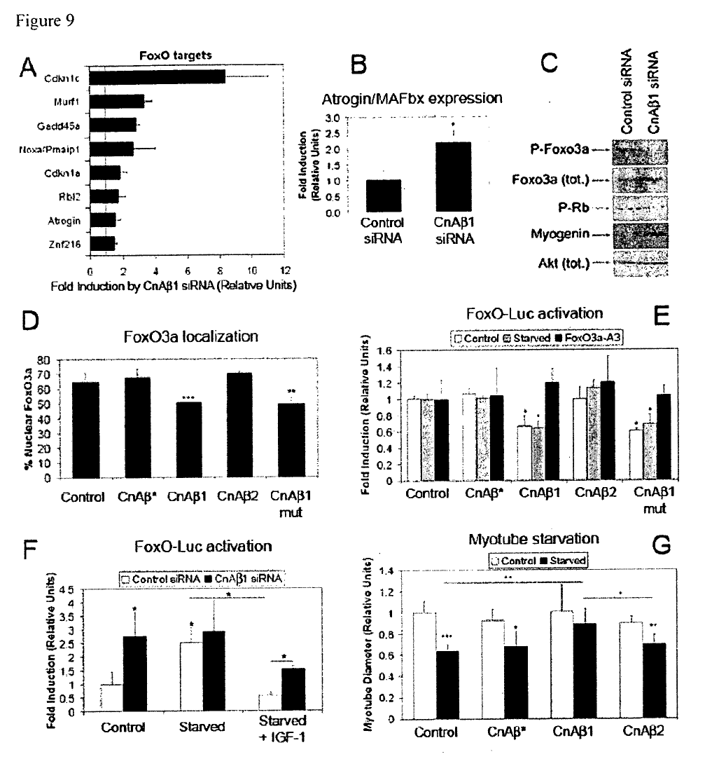

Finally, yet another aspect of the present invention relates to FoxO3, its use in screening methods, and modulators of the activity and/or expression—as well as therapeutical uses thereof—of the human transcription factor FoxO3 (and mammalian homologs thereof) in analogy to what is described for CnAβ1, since it was surprisingly found that CnAβ1 siRNA abrogated FoxO3 phosphorylation, suggesting that CnAβ1 is required to maintain FoxO in an inactive state (FIG. 9C).

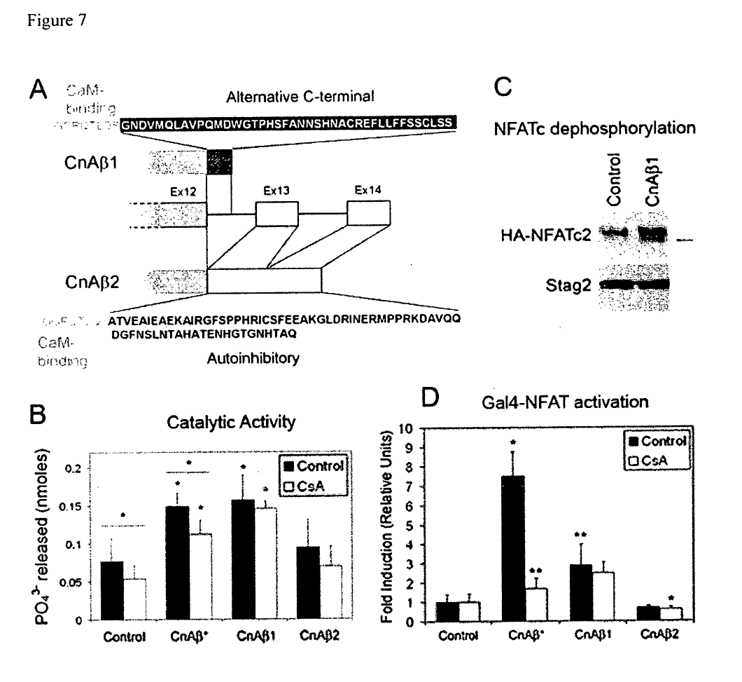

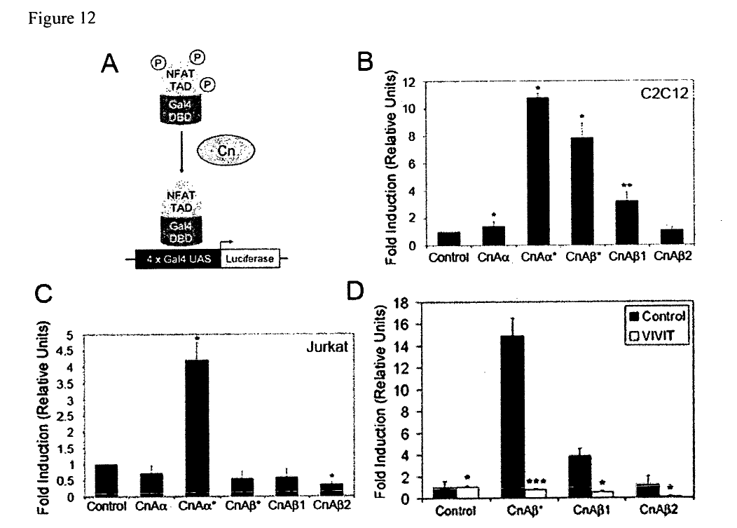

To determine whether the lack of an autoinhibitory domain would render the CnAβ1 isoform constitutively active, the inventors analyzed Cn phosphatase activity in HEK293 cells transfected with expression plasmids encoding CnAβ1, CnAβ2, or CnAβ* (in which the autoinhibitory domain has been artificially deleted). Phosphatase activity in CnAβ1 transfected cells was as high as those expressing constitutively active CnAβ*, compared to cells transfected with CnAβ2 (FIG. 7B). Co-transfection of C2C12 muscle cells with CnAβ1 and an HA-NFAT expression vectors resulted in extensive NFAT dephosphorylation (FIG. 7C). Co-transfection with different Cn constructs together with a Gal4-NFAT chimera and a Gal4-Luc reporter plasmid showed that in the absence of further stimulus, CnAβ1 activated NFAT in C2C12 muscle (FIG. 7D and FIG. 12A, 14B) but not in Jurkat T cells (FIG. 12C), albeit to a lower degree than the truncated, constitutively active CnAβ form. Interestingly, both the increased phosphatase activity and the activation of NFAT induced by CnAβ1 were resistant to the action of CsA, whereas truncated Cn forms were inhibited by the immunosuppressant drug (FIG. 7D). Conversely, over-expression of the VIVIT peptide, which blocks the interaction between Cn and NFAT prevented the activation of NFAT by all Cn forms (FIG. 12D). Taken together, these results show that CnAβ1 is a physiological form of constitutively active Cn that is insensitive to the action of CsA.

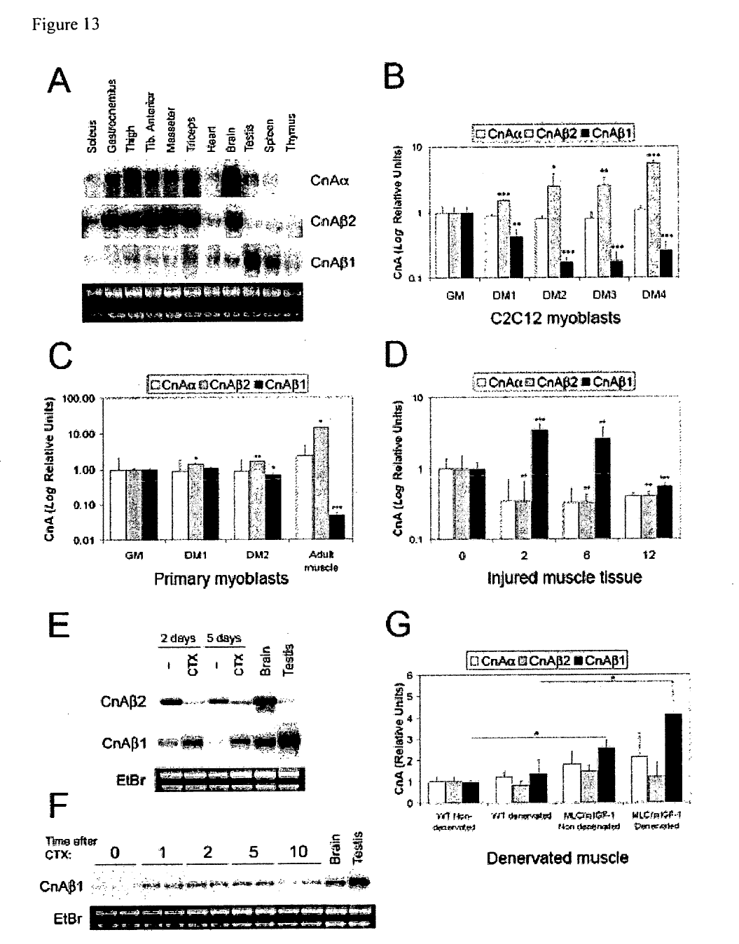

The inventors therefore analyzed the CnA expression patterns in different healthy and cardiotoxin-injured muscles by Northern blot and quantitative RT-PCR (qRT-PCR). All muscles showed high expression levels of CnAβ and CnAβ2 and weaker expression of CnAβ1 (FIG. 13A). Differentiation of primary and C2C12 myoblasts, both of which expressed high CnAβ1 levels, was accompanied by a progressive increase in CnAβ2 and a decline in CnAβ1 levels (13B, 13C). In vivo, skeletal muscle regeneration in response to injury induced a transient rise in CnAβ1 transcripts and a concomitant decrease in CnAβ2 transcripts (FIG. 13D-F). CnAβ1 expression was also induced in MLC/mIGF-1 transgenic mice especially during recovery from sciatic denervation (FIG. 13G). These results implicate the predominant CnAβ2 isoform in skeletal muscle differentiation and maturation, whereas CnAβ1 might play a role during myoblast proliferation and muscle regeneration.

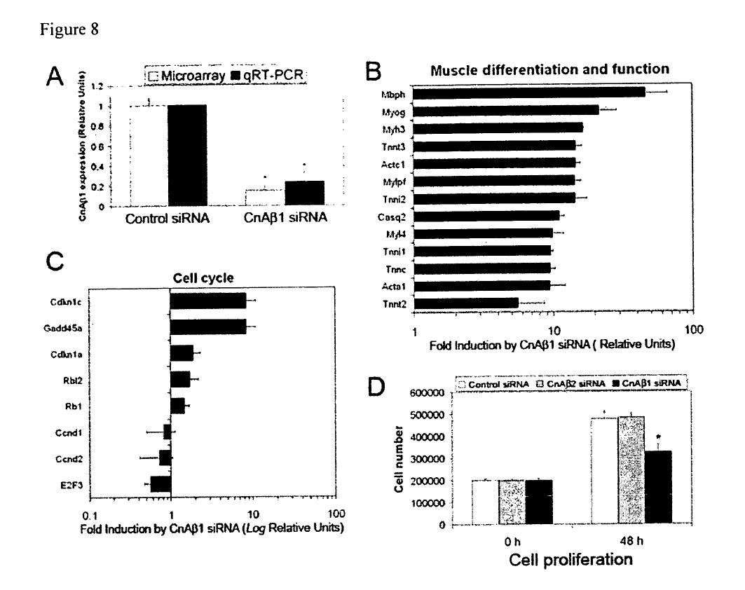

To characterize the potential function of CnAβ1, the inventors knocked-down CnAβ1 expression by transfecting proliferating C2C12 myoblasts with siRNA specific for CnAβ1 or luciferase (control) (FIG. 8A). After 48 h in growth medium, oligonucleotide-based microarray analysis showed an induction of differentiation-specific gene expression in cells transfected with the CnAβ1 siRNA, together with an increase in negative cell cycle regulators and a decrease in genes involved in cell cycle progression (FIG. 8B, 8C). A corresponding decrease in cell proliferation was observed after CnAβ1, but not CnAβ2, knock-down (FIG. 8D), indicating that CnAβ1 expression is necessary for cell cycle progression.

Many of the negative cell cycle regulators induced by CnAβ1 siRNA, such as p21, Rbl2 or Gadd45a, are known to be regulated by the FoxO family of transcription factors (O. F. Bueno, E. B. Brandt, M. E. Rothenberg, J. D. Molkentin, Proc. Natl. Acad. Sci. USA 99, 9398 (2002). G. Dobrowolny et al., J. Cell Biol. 168, 193 (2005). J. Seoane, H. V. Le, L. Shen, S. A. Anderson, J. Massague, Cell 117, 211 (2004). Y. Furukawa-Hibi, K. Yoshida-Araki, T. Ohta, K. Ikeda, N. Motoyama, J. Biol. Chem. 277, 26729 (2002)). Additional FoxO target genes were induced after CnAβ1 knock-down (FIG. 9A), accompanied by increased expression of atrogenes Murfl and MAFbx/Atrogin (FIG. 9B) that are known to mediate FoxO-induced muscle atrophy (M. Sandri et al., Cell 117, 399 (2004). T. N. Stitt et al., Mol. Cell 14, 395 (2004)). CnAβ1 siRNA abrogated FoxO3a phosphorylation at Ser253, suggesting that CnAβ1 is required to maintain FoxO in an inactive state (FIG. 9C). Corresponding decreased phosphorylation of Rb and increased expression of myogenin was consistent with cell cycle arrest and myoblast differentiation observed in the microarray analysis of CnAβ1 siRNA-transfected cells (FIG. 9C). Moreover, overexpression of CnAβ1, but not CnAβ2 or the constitutively active CnAβ*, in C2C12 cells inhibited FoxO nuclear localization (FIG. 9D) and the activity of a FoxO-dependent luciferase reporter (FIG. 9E), whereas CnAβ1 siRNA activated the reporter (FIG. 9F). Interestingly, FoxO induction of MAFbx/Atrogin has recently been shown to inhibit Cn activity (Y. G. Ni et al., Circulation 114, 1159 (2006). H. H. Li et al., J. Clin. Invest. 114, 1058 (2004), suggesting that inhibition of FoxO by CnAβ1 might also contribute to the increased Cn activity in CnAβ1-expressing cells.

To determine whether FoxO inhibition by CnAβ1 can prevent myotube atrophy, C2C12 stable transfectants overexpressing CnAβ*, CnAβ1 or CnAβ2 were differentiated into myotubes for 8 days and then starved for 16 h. As shown in FIG. 9G, starvation caused a 35-40% decrease in myotube diameter that was prevented by CnAβ1, but not by CnAβ* or CnAβ2. Accordingly, CnAβ1 overexpression repressed FoxO in starving cells (FIG. 9E) and CnAβ1 knockdown blocked repression of FoxO by IGF-1 (FIG. 9F). However, CnAβ1 had no effect on a constitutively active FoxO3a-A3 mutant lacking the three Akt/SGK phosphorylation sites (J. Nakae, V. Barr, D. Accili, EMBO J. 19, 989 (2000)) (FIG. 9E), which also renders it insensitive to IGF-1 (W. H. Zheng, S. Kar, R. Quirion, J. Biol. Chem. 275, 39152 (2000). A. Brunet et al., Mol. Cell. Biol. 21, 952 (2001)). Since CnAβ1 expression is induced by IGF-1 in regenerating muscle (G. Dobrowolny et al., J. Cell Biol. 168, 193 (2005)). and FIG. 13G) IGF-1 and CnAβ1 may share common pathways for the inhibition of FoxO and FoxO-regulated processes. Interestingly, inhibition of FoxO activity and nuclear localization by CnAβ1 did not require a functional phosphatase domain, since a phosphatase-dead CnAβ1 mutant (CnAβ1mut) also reduced FoxO nuclear localization and activation (FIG. 9D, 9E). In addition, NFAT-regulated genes were only slightly affected by CnAβ1 knock-down in myoblasts.

These results suggest that the main role of CnAβ1 in proliferating myoblasts is to inhibit FoxO, allowing cell proliferation and preventing differentiation, rather than to activate NFAT, which is involved in myoblast differentiation (U. Delling et al., Mol. Cell. Biol. 20, 6600 (2000).) after CnAβ1 expression has been shut down.

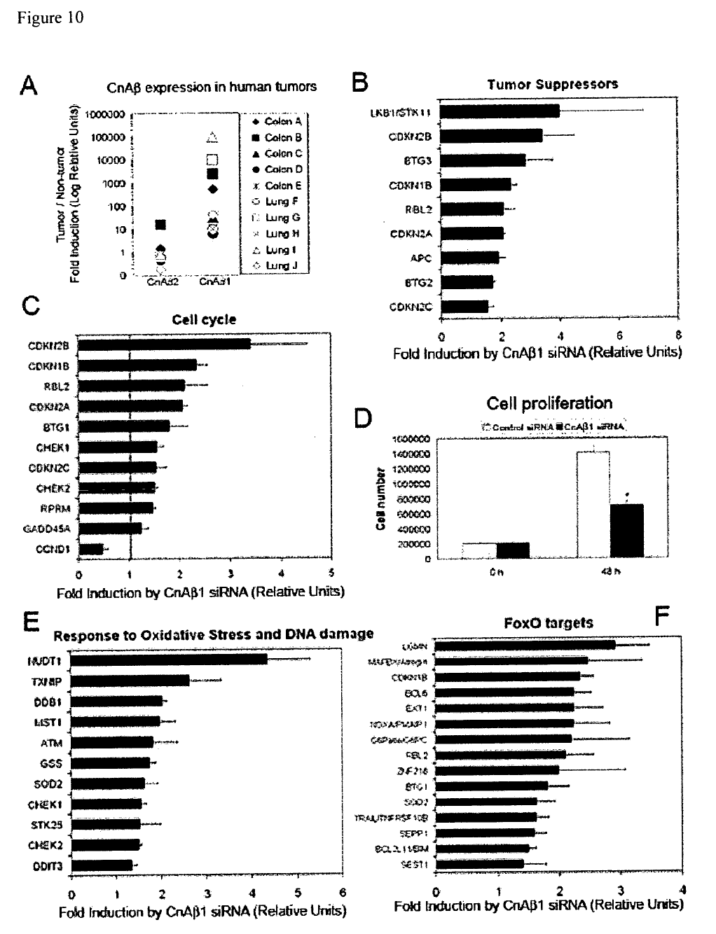

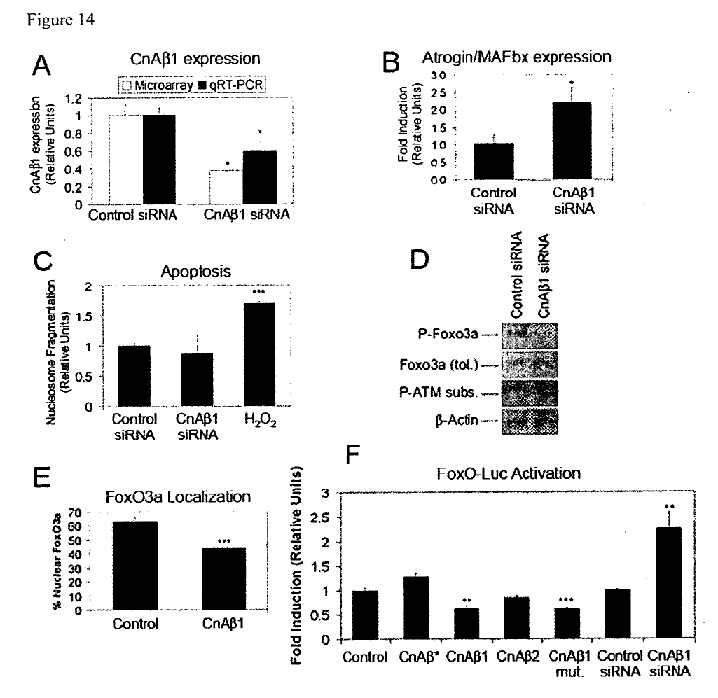

Given the requirement of CnAβ1 for cell proliferation in muscle cultures, the inventors analyzed the expression of CnAβ1 and CnAβ2 in tumor tissue and matching non-tumor tissue from 5 human lung tumor and 5 colon tumor patients. A strong increase in CnAβ1 expression was observed in tumor vs. non-tumor tissue, whereas the expression of CnAβ2 barely changed in most patient samples (FIG. 10A). CnAβ1 siRNA-mediated knock-down of the normally high CnAβ1 expression levels in HEK293 tumor cells (FIG. 14A) induced the expression of tumor suppressor genes and negative regulators of the cell cycle (FIG. 10B, 10C) and reduced proliferative capacity (FIG. 10D).

Surprisingly, CnAβ1 knock-down increased expression of many genes involved in the oxidative stress and DNA damage response (FIG. 10E). Since FoxO has been shown to be necessary and sufficient for the induction of this response (Y. Furukawa-Hibi, K. Yoshida-Araki, T. Ohta, K. Ikeda, N. Motoyama, J. Biol. Chem. 277, 26729 (2002). H. Tran et al., Science 296, 530 (2002). G. J. Kops et al., Nature 419, 316 (2002). M. K. Lehtinen et al., Cell 125, 987 (2006), the inventors studied whether CnAβ1 inhibited FoxO activity in tumor cells as it does in myoblasts. Indeed, transfection of HEK293 cells with CnAβ1 siRNA revealed an increase in the expression of FoxO targets, including p27KIP1/CDKN1B and MAFbx/Atrogin (FIG. 10F, FIG. 14B) which was not associated with cell death (FIG. 14C). Activation of FoxO in tumor cells by CnAβ1 knock-down was further confirmed by western blot, which showed decreased FoxO3a phosphorylation accompanied by the activation of the DNA damage response regulator ATM (FIG. 14D) and induction of a FoxO-dependent reporter (FIG. 14F). Overexpression of either CnAβ1 or the phosphatase-dead CnAβ1mut decreased the percentage of cells with nuclear FoxO3a and inhibited the FoxO-dependent reporter (FIG. 14E, 14F), whereas other CnA isoforms had no effect on FoxO activity. Although CnAβ1 is unlikely to be sufficient for tumor development, the inventors' results unveil a key role for this Cn splicing variant in tumor progression.

In summary, in the context of the present invention, the inventors have shown that CnAβ1, a naturally occurring, CsA-insensitive Cn splicing variant, inhibits FoxO independently of its phosphatase activity. This inhibition, specific for the CnAβ1 isoform, uncovers an unexpected connection between the FoxO and calcineurin pathways and may have important implications for the regulation of cell proliferation, tumor development, muscle wasting and aging. In this regard, the inventors show that CnAβ1 is necessary for myoblast proliferation and represses myotube atrophy. In addition, CnAβ1 is strongly induced in tumors, where it supports cell proliferation, represses tumor suppressor genes and prevents the onset of the oxidative stress response, thus favoring tumor progression. CnAβ1 may be also play a role cell proliferation during development, since its expression is high in ES cells and developing tissues and transgenic mice constitutively over-expressing a CnAβ1 miRNA are embryonic lethal. These results establish CnAβ1 as a complex modulator of both physiological and pathological processes, and opens new roads for the development of novel therapeutic strategies, as described and provided herein.

The above and other features, objects and advantages of the present invention will be better understood by a reading of the specification in conjunction with the following examples and Figures without being limited thereto. In the Figures and sequences,

SEQ ID No. 1 shows the nucleotide sequence of CnAβ1.

SEQ ID No. 2 shows the amino acid sequence of CnAβ1.

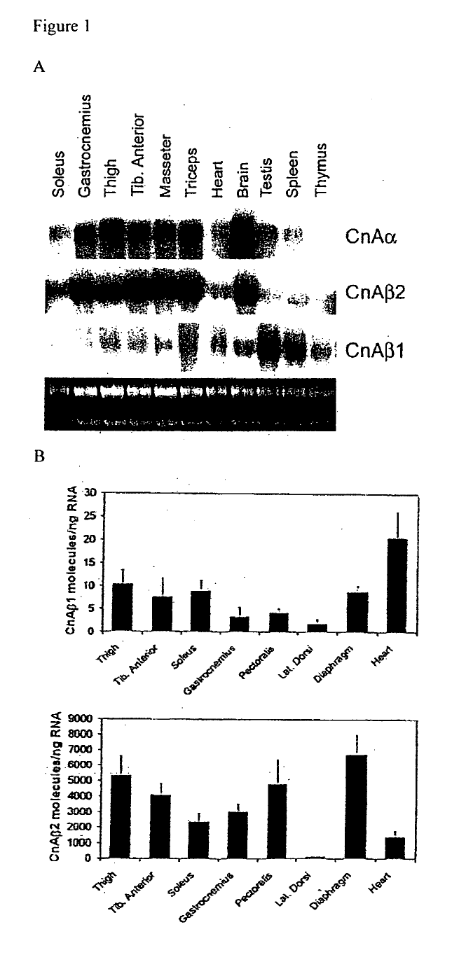

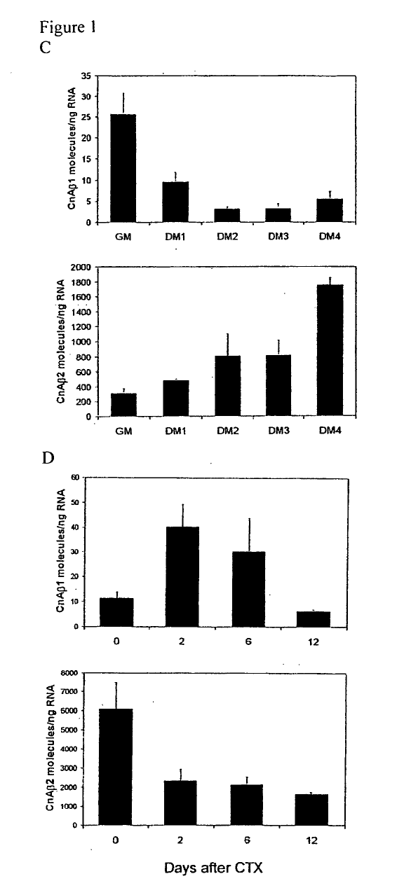

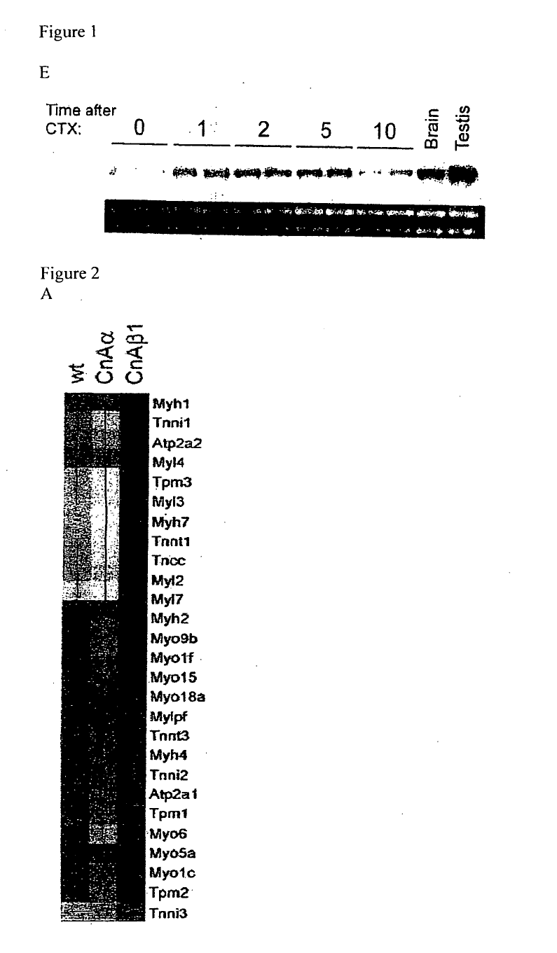

FIG. 1 shows that CnAβ1 is induced during skeletal muscle regeneration. The expression pattern of the different CnA isoforms was analyzed in different striated muscles by northern blot (A) and qRT-PCR (B). (C) Expression of the two CnAβ variants was determined in C2C12 cells incubated in differentiation medium (DM) for different days. (D) Two month-old wild type mice were injured in the quadriceps by cardiotoxin injection and allowed to recover for 2, 6 or 12 days (0, uninjured). Total RNA was extracted and the expression of CnAβ1 and CnAβ2 determined by qRT-PCR. E. The induction of CnAβ1 during skeletal muscle regeneration was confirmed by northern blot analysis.

FIG. 2 shows that CnAβ1 induces an increase in slow-type muscle fibers. (A) Total RNA was extracted from the quadriceps of 2-month old MLC-CnAβ1, MLC-CnAα and wild type mice and subjected to microarray analysis. The changes in the expression levels of different muscle fiber molecules are shown over the wild type mice value, represented by red (increase) or green colors (decrease). (B) The induction in vivo of TnI-slow mRNA expression by CnAβ1, but not CnAα, was confirmed by qRT-PCR. Results are given as relative units referred to the values obtained in the wild type mouse quadriceps.

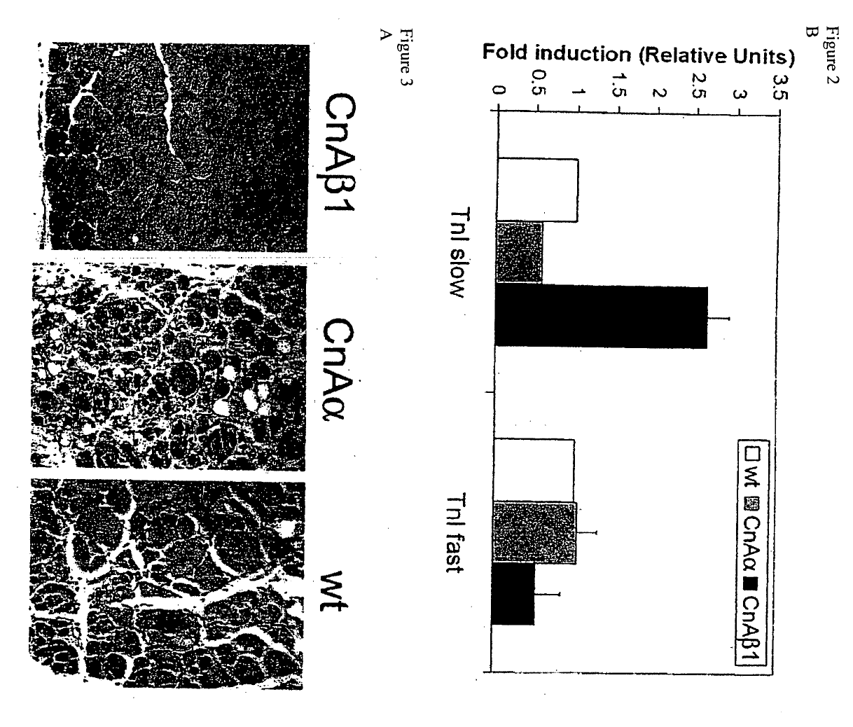

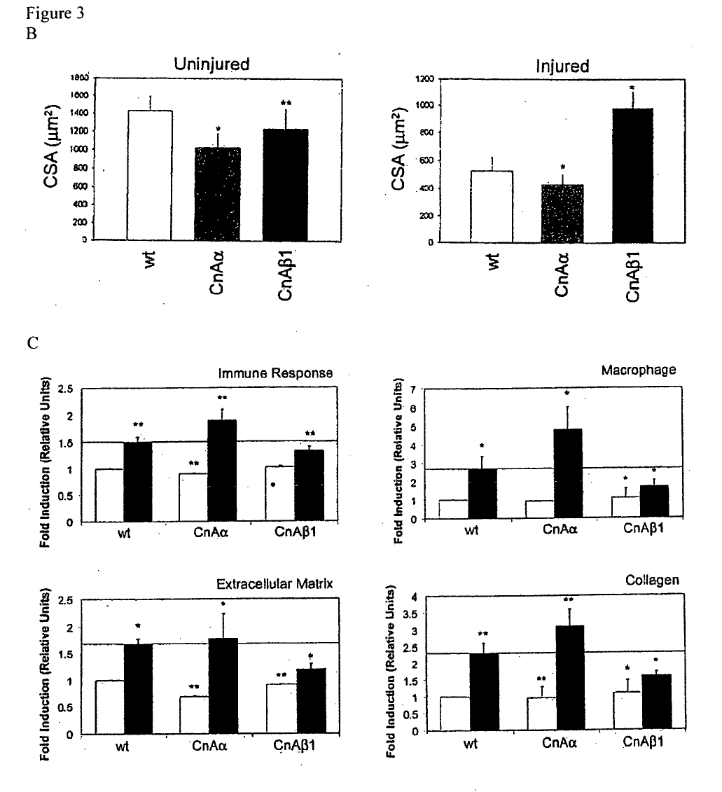

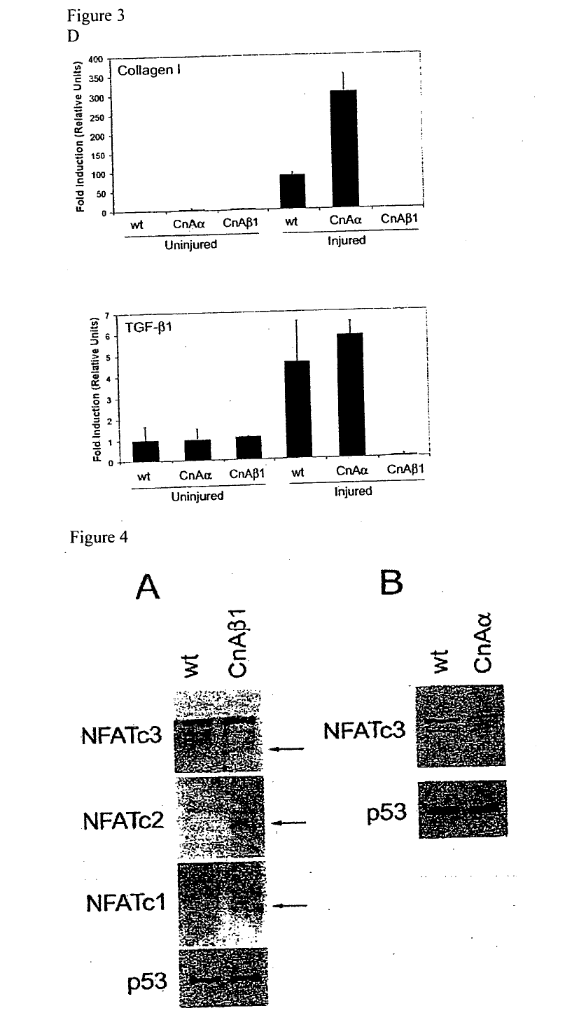

FIG. 3 shows that CnAβ1 induces skeletal muscle regeneration. The quadriceps of WT, MLC-CnAα and MLC-CnAβ1 mice 12 days after cardiotoxin injection was analyzed by HandE staining (A) and the average cross sectional area (CSA) of the regenerating myotubes was measured (B) Results are expressed as μm²±SD. * P<0.005, ** P<10⁻¹². (C) Microarray analysis of the quadriceps from WT, MLC-CnAα and MLC-CnAβ1 mice uninjured and 12 days after injury. Average expression ±SD of the genes involved in different biological processes is shown in injured black bars) and uninjured mice (white bars), referred to the uninjured wild type mouse. * P<0.01; ** p<10⁻⁷. (D) Samples in (C) were analyzed by qRT-PCR for the presence of collagen I and TGF-β expression.

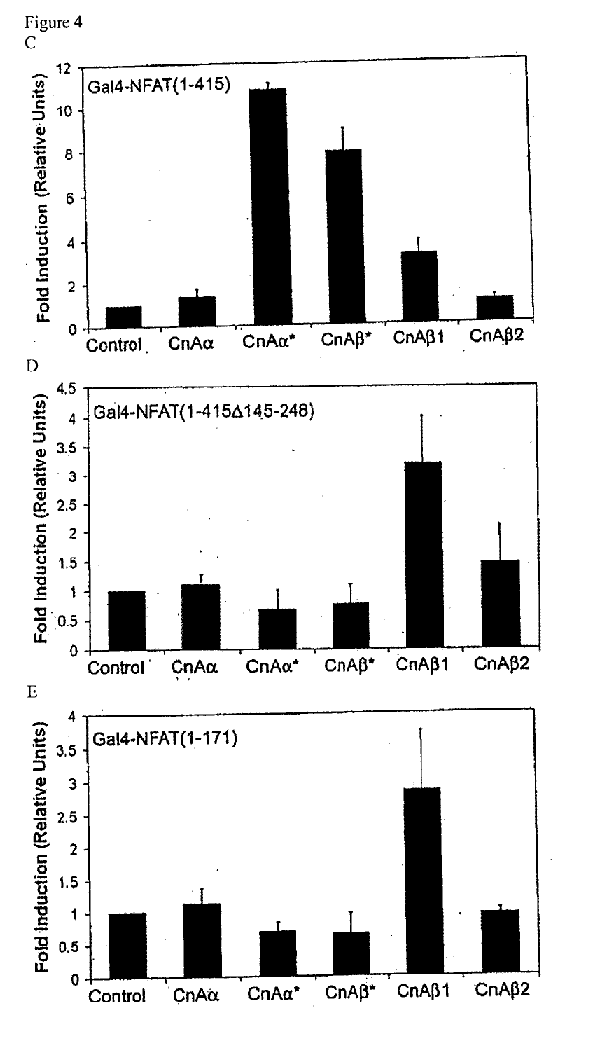

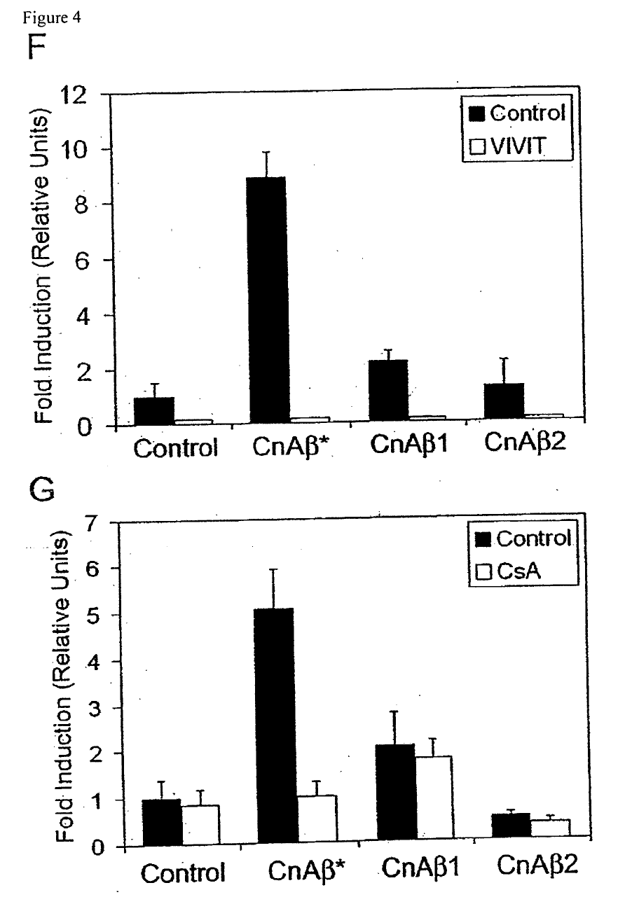

FIG. 4 shows that CnAβ1 constitutively activates NFAT. (A) Nuclear extracts from WT and MLC CnAβ1 mice were analyzed for the presence of NFATcI, NFATc2 and NFATc3 by western blot (A). The extra-dephosphorylated NFATc band (arrow) was not observed in MLC-CnAα mice (B) Equal loading of the different samples was demonstrated by an anti-p53 antibody. C-E, C2C12 cells were transiently transfected with the different CnA expression vectors (control, empty pcDNA3), the Gal4-Luc reporter plasmid and pGal4-MAT vectors, bearing the Gal4 DNA-binding domain linked to different NFATc regions (aa indicated in brackets). Results show fold induction over the control value and represent the average of at least three independent experiments. (F), (G) C2C12 cells were transfected as in (C) along with pGFPVMT (F). Where indicated, 10 ng/ml cyclosporin A was added to the culture after stopping transfection (G). Results are represented as in (C).

FIG. 5 shows the analysis of the signaling pathways activated by CnAβ1. Diagram of the protein kinases activated by MLC-CnAβ1 (A) in uninjured quadriceps. (B), (C) Diagram of the kinase pathways activated 12 days after cardiotoxin injury in the quadriceps of WT (B) and MLC CnAβ1 (C) Yellow, no activation; red, activation; green, inhibition; white, not assayed or no signal detected. All values compared to uninjured WT. Different shades of color indicate different intensity of activation as observed.

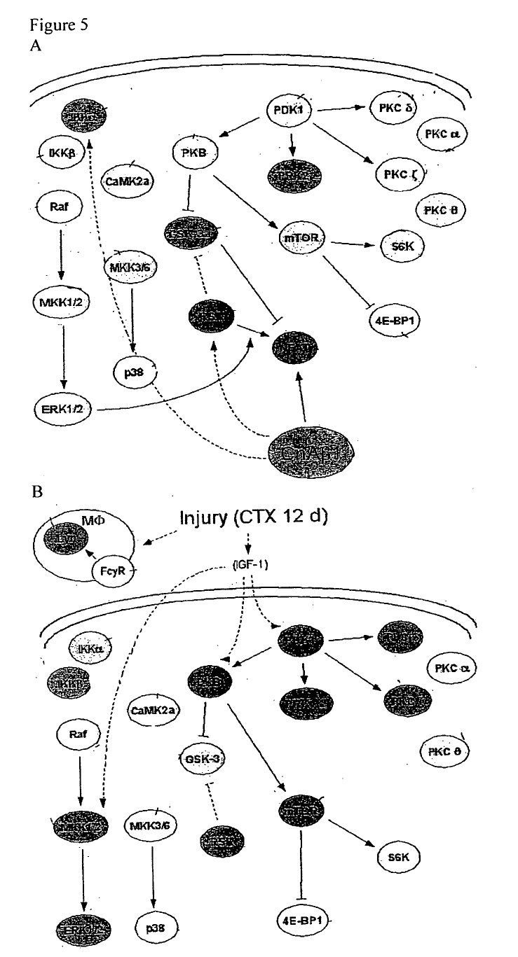

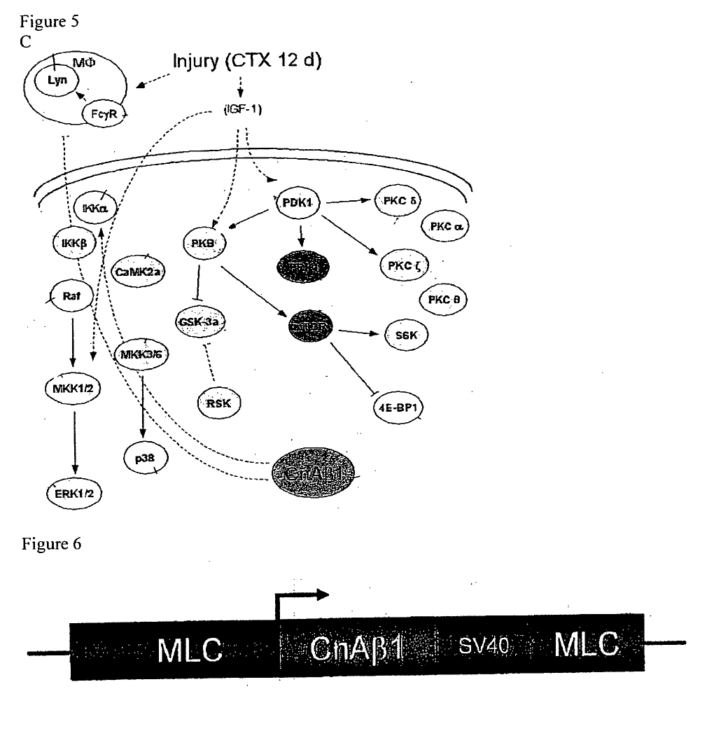

FIG. 6 shows the transgenic construct used in the generation of MLC-CnAβ1 and MLC-CnAα mice. CnAβ1 and CnAα genes where placed under the control of the myosin light chain promoter and enhancer, which allows skeletal muscle-specific, postmitotic expression of CnAβ1 and CnAα. The MLC-CnAβ1 construct is shown.

FIG. 7 shows that CnAβ1 is a constitutively active Cn isoform. (A) Schematic diagram of CnAβ1 and CnAβ2 isoforms, alternative splicing variants of the CnAβ gene. CnAβ1 includes an alternate C-terminal domain encoded by intronic sequences (above) whereas CnAβ2 includes a canonical autoinhibitory domain encoded by exons 13-14 (below) (B) HEK293 cells were transfected with CnA expression vectors or empty pcDNA3.1 (control) and Cn phosphatase activity was assayed in the absence (black bars) or presence (white bars) of 1 μg/ml CsA. (C) C2C12 myoblasts were transiently co-transfected with a HA-NFATc2 expression vector and pcDNA3.1-CnAβ1 or empty pcDNA3.1. After 2 days in DM, nuclear extracts were analyzed by western blot using an anti-HA antibody. Anti-Stag2 shows equal nuclear protein loading. Arrow indicates increased dephosphorylated NFAT. (D) C2C12 myoblasts were transiently transfected with CnA expression vectors (or empty pcDNA3.1 as a control), the pGal4-Luc reporter and pGal4-NFAT-1-415 and grown in DM for 2 days. Where indicated 1 μg/ml cyclosporin A (white bars) or EtOH as a vehicle (black bars) was added to the culture after transfection. Results show fold induction over the control value ±SD and represent the average of at least three independent experiments. * P<0.05; ** P<0.005.

FIG. 8 shows that a CnAβ1 knock-down blocks myoblast proliferation and induces differentiation. (A) C2C12 myoblasts were transfected with CnAβ1-specific or control siRNA, grown for 48 h in growth medium (GM) and the expression of CnAβ1 was assayed by microarray and qRT-PCR. (B), (C) RNA was extracted from myoblasts transfected with control or CnAβ1 siRNA and subjected to microarray analysis. Genes involved in myoblast differentiation (B) and cell cycle ((C), note log units) are shown as fold induction ±SD of values from cells transfected with CnAβ1 siRNA over those transfected with control siRNA. (D) C2C12 myoblasts were transfected with siRNA specific for CnAβ2, CnAβ1 or a control and cell proliferation was assayed 0 and 48 h after transfection. * P<0.05.

FIG. 9 shows that CnAβ1 inhibits FoxO. (A)-(C), C2C12 myoblasts were transfected with control or CnAβ1 siRNA and grown for 48 h in GM. (A) RNA was analyzed by microarray for induction of FoxO targets. (B) Induction of MAFbx/Atrogin mRNA expression was confirmed by qRT-PCR. (C) Phosphorylation status of FoxO3a and Rb was analyzed by western blot together with the expression of total FoxO3a, Myogenin and Akt. (D) C2C12 myoblasts were transfected with pGFP-FoxO3a and CnA expression vectors and nuclear GFP-Foxo3a was quantified. (E), (F), C2C12 myoblasts were co-transfected with the p6xDBE-Luc reporter and control or different CnA expression vectors ((E), note each condition is represented independently) or CnAβ1 siRNA (F) and incubated for 48 h in GM (Control) or starving conditions. FoxO3a-A3 vector (E) or 25 ng/ml IGF-1 (F) were added where indicated. Luciferase activity is expressed as fold induction ±SD over the value of empty pcDNA3.1 for the three conditions (E) or control siRNA in GM (F). (G) C2C12 myoblasts stably transfected with the different CnA or empty (control) expression vectors were differentiated for 8 days and incubated overnight in starving conditions. The myotube diameter was determined and expressed as relative units ±SD. * P<0.05; ** P<0.005; *** P<0.0005.

FIG. 10 shows that CnAβ1 is expressed in tumors. (A) CnAβ2 and CnAβ1 mRNA expression was determined by qRT-PCR in five human lung and colon tumors. Results are expressed as fold induction in tumor tissue over non-tumor tissue in each patient. (B), (C), (E), (F), HEK293 tumor cells were transfected with control or CnAβ1 siRNA, and RNA was analyzed after 48 h by microarray. Induction of tumor suppressor genes (B), negative cell cycle regulators (C), genes involved in the oxidative stress and DNA damage response (E) and FoxO targets (F) is shown as values from cells transfected with CnAβ1 siRNA over those transfected with control siRNA. (D) HEK293 cells were transfected with control or CnAβ1 siRNA and cell proliferation was assayed 0 and 48 h after transfection. * P<0.05.

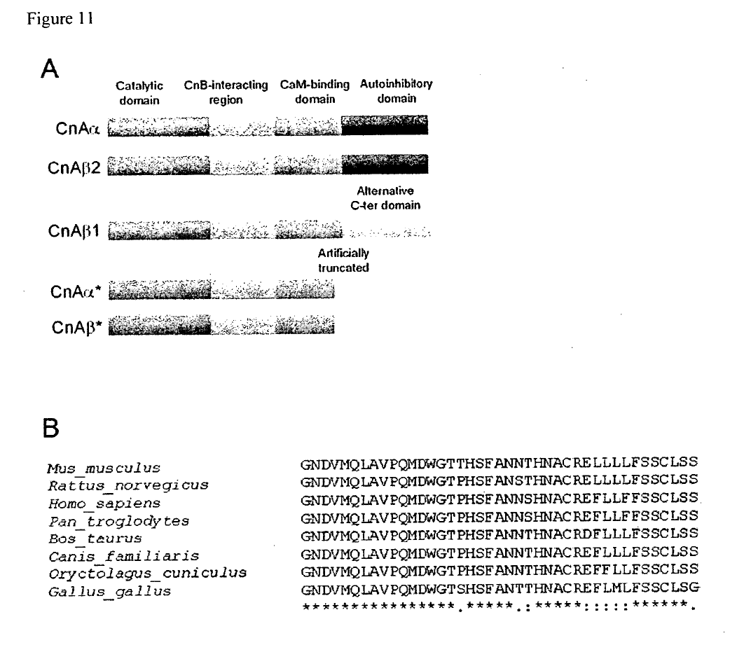

FIG. 11 shows that the C-terminal domain of CnAβ1 is conserved among vertebrates. (A), Schematic representation of CnA isoforms. CnAα, CnAβ2 and CnAγ share a catalytic domain, a CnB-interacting region, a CaM-binding domain and an autoinhibitory domain. CnAβ1 is a naturally-occurring splicing variant of CnAβ2, with alternative C-terminal domain that has no homology with the autoinhibitory domain. CnAα* and CnAβ* represent constitutively activated CnA isoforms in which the autoinhibitory domain has been artificially deleted. (B) Alignment of the CnAβ1 C-terminal amino acid sequence in different species obtained by translation of intron 12-13.

FIG. 12 shows that CnAβ1 constitutively activates NFAT in C2C12 cells in a VIVIT-sensitive manner. (A) Schematic diagram showing Cn activation of the Gal4-NFATc chimera used in these experiments. (B) C2C12 myoblasts were transfected with expression vectors for Gal4-NFAT chimera and Cn isoforms together with a pGal4-Luc reporter plasmid. Luciferase activity was analyzed after 48 h in DM and expressed as fold induction ±SD over control vector. (C) Jurkat T cells were transfected with expression vectors for Gal4-NFAT chimera and Cn isoforms together with a pGal4-Luc reporter plasmid. Luciferase activity was analyzed after 48 h as in (B). (D) C2C12 myoblasts were transfected as in (B) together with a VIVIT expression plasmid or control vector and luciferase activity was analyzed as in B. *P<0.05, **P<0.005, ***P<0.0005.

FIG. 13 shows that CnAβ1 is expressed in proliferating myoblasts and regenerating skeletal muscle. (A), Expression patterns of CnAα, CnAβ2 and CnAβ1 isoforms were analyzed in different striated muscles by northern blot. (B), CnA isoform expression was determined by qRT-PCR in C2C12 myoblasts incubated in growth medium (GM) or differentiation medium (DM) for days indicated. Results are expressed as fold induction over the value in GM (note: Log relative units ±SD). (C), Comparison of CnA isoform expression in proliferating primary myoblasts and adult skeletal muscle. Satellite cells were isolated using the single fiber technique from rat EDL, induced to proliferate in GM and then shifted to DM for days indicated. RNA was extracted and CnAα, CnAβ2 and CnAβ1 expression analyzed by qRT-PCR. Results are expressed as fold induction over myoblasts in GM (note Log relative units ±SD). (D) Two month-old wild type mice were injured in the quadriceps muscle by cardiotoxin injection and allowed to recover for 2, 6 or 12 days (0, uninjured). Total muscle RNA was extracted and CnAα, CnAβ2 and CnAβ1 expression was determined by qRT-PCR. Results are expressed as fold induction over uninjured muscle (note Log relative units ±SD). (E), (F) The switch between CnAβ1 and CnAβ2 expression during skeletal muscle regeneration and the transient increase in CnAβ1 mRNA expression were confirmed by northern blot. (G), CnA isoform expression in denervated muscle from wild type and MLC/mIGF-1 transgenic mice was analyzed by qRT-PCR 28 days after denervation and expressed as fold induction ±SD over non-denervated wild type. * P<0.05; ** P<0.005; *** P<0.0005.

FIG. 14 shows that CnAβ1 inhibits FoxO in HEK293 cells. (A) HEK293 cells were transfected with control or CnAβ1 siRNA, grown for 48 h and CnAβ1 expression was analyzed by qRT-PCR and microarray. (B) Cells were transfected as in A and MAFbx/Atrogin expression was determined by qRT-PCR. (C) HEK293 cells were transfected as in A and apoptotic activity was determined 48 h later. 10 mM H₂O₂ stimulation for 2 h was used as a positive control. (D) The phosphorylation status of FoxO3a and ATM substrates was analyzed by western blot together with the expression of total FoxO3a and β-Actin. (E) HEK293 cells were transfected with a GFP-FoxO3a and Cn expression vectors and the % of cells with nuclear GFP-Foxo3a was quantified. (F) HEK293 cells were transfected with p6xDBE-Luc reporter together with control or CnAβ1 siRNA or different Cn expression vectors and luciferase activity analyzed 48 h later. Luciferase activity is expressed as fold induction over the value of the empty pcDNA3.1 (Control). * P<0.05; ** P<0.005; *** P<0.0005.

### EXAMPLES

### Methods

### Transgenic Mice

MLC-CnAβ1 and MLC-CnAα mice carry the CnAβ1 and CnAα genes respectively under the control of the myosin light chain promoter and enhancer, which allows skeletal muscle specific, post-mitotic expression of CnAβ1 and CnAα. FVB mice were used as embryo donors and the transgenic subjects were generated using standard methods. Positive founders were subsequently bred to FV3 wild-type mice and MLC-CnAα and MLC-CnAβ1 transgenic mice were selected by PCR using tail digests. The subjects were housed in a temperature-controlled (22° C.) room with a 12:12 h light-dark cycle.

DNA Isolation, Northern Blot, Real Time PCR, siRNA, RNA Isolation, and cDNAs

The following stealth siRNA (Invitrogen) were used:

mouse CnAβ1
(AGUUCCUGUCUUAGCAGCUGACAUA),
human CnAβ1
(GAUAUGGGAGCAGCUCAUAUCAUAA),
mouse and human CnAβ2
(GACUGGCAACCAUAGUGCCCAGUGA),
and
Luciferase, used as a negative control
(GCCCGCGAACGAGAUUUAUAAUGAA).

Total RNA was extracted from snap-frozen tissue or cell cultures by using Trizol (Sigma, S. Louis, Mo.). 20 μg total RNA were resolved by agarose gel, blotted and hybridized with CnAα-, CnAβ2- and CnAβ1-specific probes. For real-time PCR analysis, cDNA was generated using a Roche kit (Roche, Mannheim, Germany) from 2 μg of total RNA in a 40 μl volume. Human tumor cDNA (matching tumor and non-tumor samples from the same patient) from 5 lung tumor patients and 5 colon tumor patients was purchased from Clontech (Mount View, Calif.).

A total of 2 μl of the cDNA reaction were amplified by PCR in duplicate by using a SYBR green kit (Finnzymes, Espoo, Finland) and the following annealing temperatures and primers:

CnAβ1, 57°
(fwd-AGAAGGTGAAGACCAGT,
rev-AGCAAGTTGCATAACATCATT),
CnAβ32 58°
(fwd-AGGCTATTGAGGCTGAAA,
rev-CGGATCTCAGAAAGCAC),
CnAα, 57°
(fwd-CAAGGCGATTGATCCCA,
rev-TCGAAGCACCCTCTGTTA),
CnB I
(fwd 5′-ATGCAGATAAGGATGGA-3′,
rev-5′-GAAAGCAAAAGTGTTGGG-3′),
ubiquitin B, 58°
(fwd-5′-TGGCTATTAATTATTCGGTCTGCAT,
rev-GCAAGTGGCTAGAGTGCAGAGTAA),
TnI-slow, 55°
(fwd-5′-TGCTGAAGAGCCTGATGCTA-3′,
rev 5′-GAACATCTTCTTGCGACCTTC-3′),
TnI-fast, 55°
(fwd-5′-GAAGGAGAACTACCTGTCAGA-3′,
rev-5′-TGGGCAGTTAGGACTCAGACTC-3′).

MAFbx/Atrogin primers have been previously described (F. Mourkioti et al., J. Clin. Invest. In press. (2006)). Data were normalized with ubiquitin B values and expressed as fold induction over a control sample.

Total RNA was extracted from snap-frozen tissue or cell cultures by using Trizol (Sigma, S. Louis, Mo.). 20 pg of total RNA were resolved in an agarose gel, blotted and hybridized with a CnAα-, CnAβ2- and CnAβ1-specific probe. For the real-time PCR analysis, cDNA was generated using a Roche kit (Roche, Mannheim, Germany) from 2 μl of total RNA in a 40 μl volume. 2 μl of the cDNA reaction were amplified by PCR in triplicate by using a SYBR Green kit (Finnzymes, Espoo, Finland) and the following annealing temperatures and primers:

CnAβ1, 57°
(fwd-AGMGGTGAAGACCAGT,
rev-AGCAAGTTGCATAACATCATT),
CnAβ2, 58°
(fwd-AGGCTATTGAGGCTGAAA,
rev-CGGATCTCAGMGCAC),
CnAα, 57°
(fwd-CAAGGCGATTGATCCCA,
rev-TCGAAGCACCCTCTGTTA),
CnB1
(fwd 5′-ATGCAGATAAGGATGGA-3′,
rev-5′-GAAAGCAAAAGTGTTGGG-3′),
ubiquitin ligase, 58°
(fwd-5′-TGGCTATTAATTATTCGGTCTGCAT,
rev-GCAAGTGGCTAGAGTGCAGAGTU),
TnI-slow, 55°
(fwd-5′-TGCTGAAGAGCCTGATGCTA-3′,
rev 5′-GAACATCTTCTTGCGACCTTC-3′),
TnI-fast, 55°
(fwd-5′-GAAGGAGAACTACGTGTCAGA-3′,
rev-5′-TGGGCAGTTAGGACTCAGACTC-3′).

Data were normalized with the ubiquitin ligase values and expressed as fold induction over a control sample. For the CnA isoforms, normalized values were quantified by using a standard curve of known plasmid concentration and expressed as number of molecules per ng of total RNA.

### Muscle Injury and Regeneration

Tibialis anterior (TA) and quadriceps muscles from 2-month MLC-CnAβ1, MLC-CnAα or wild type mice were given five (TA) or eight (quadriceps) 5 μl injections of 10 μM cardiotoxin in PBS. TA muscles were collected 2, 6 and 12 d (2, 5 and 10 d for the northern blot) after the cardiotoxin injection, embedded in paraffin and serial sections were prepared and stained with hematoxilin and eosin (HandE). Quadriceps were collected at the same time as the TA and snap-frozen in liquid nitrogen. The cross-sectional area of myotubes was calculated in areas of active regeneration in the cardiotoxin-injured and control TA, 12 days after injury, by using the Scion Image software (www.scioncorp.com). Only fields of active regeneration close to the wound were chosen. At least 8 different sections and 1000 fibers were counted for each experimental point. All experiments involving subjects were approved by the EMBL Subject Ethics Committee and were performed according to local and institutional guidelines.

### Microarray Analysis and Sequence Comparison

Total RNA from the different samples was hybridized in triplicate on Affymetrix mouse MG_{—}430A2.0 or the human HG-U133A2.0 oligonucleotide-based arrays. Raw data were normalized and analyzed using GeneSpring-7.2 (Silicon Genetics, www.silicongenetics.com). After chip normalization to the 50^{th} percentile, samples were normalized to cells transfected with control siRNA and ANOVA statistical analysis was performed. Statistically significant genes were subjected to gene tree hierarchical clustering and lists of up-regulated and down-regulated genes were compared to annotated gene ontology lists, choosing those lists with at least 10 genes in common and a P value <10⁻¹¹ (wild type) or P<10⁻⁷ (MLC-CnAβ1). The average of the relative gene expression variation of the genes in these lists (mitochondria, 661 genes; immune response, 200 genes; macrophage-associated, 6 genes; extracellular matrix, 164 genes; collagen, 26 genes) in the different experimental conditions was calculated and expressed as fold induction over the value of the uninjured wild type mice.

Intron sequences obtained from Ensemble (www.ensemble.org) were translated using the Expasy translation engine (www.expasy.org) and compared using ClustalW (www.ebi.ac.uk/clustalw).

### Cells, Plasmids and Transfection

Cells: The murine myoblast cell line C2C12 was propagated in growth medium (GM) (DMEM 4.5 g/l glucose, 10% FCS, 2 mM L-glutamine, 5 mM Penicillin/Streptomycin) and induced to differentiate in differentiation medium (DM) (growth medium, 0% FCS, 2% horse serum). C2C12 cells were starved by incubating in PBS 1 mM CaCl₂, 1 mM MgCl₂ for 16 h as described (M. Sandri et al., Cell 117, 399 (2004)). Muscle satellite cells were isolated from rat EDL muscles using the single fiber technique as previously described (K. Suzuki et al., Circulation 104, 1207 (2001)). HEK293 cells were grown in DMEM 1 g/l glucose, 10% FCS, 2 mM L-glutamine, 5 mM Penicillin/Streptomycin. Jurkat T cells were grown in RPMI, 10% FCS, 2 mM L-glutamine, and 5 mM Penicillin/Streptomycin.

Plasmids: The expression vectors pcDNA3-CnAβ1, pcDNA3-CnAβ1mut, pcDNA3-CnAβ2, pcDNA3-CnAα, pcDNA3.1-CnAα*, pcDNA3.1-CnAβ* carry cDNAs of the respective CnA isoforms under the control of a CMV promoter (CnAα* and CnAβ* represent the artificially truncated forms of CnAα and CnAβ2; CnAβ1mut carries a D130N mutation and lacks phosphatase activity). pGal4-Luc and p6xDBE-Luc express the luciferase reporter gene under the control of three tandem binding sites for the yeast transcription factor Gal4 or six tandem binding sites for the C. elegans FoxO homologue Dafl6, respectively. pGal4-NFAT (1-415) expresses a chimerical protein containing the Gal4 DNA-binding domain linked to NFAT transcription activation and regulatory domains (amino acids 1-415) (C. Luo, E. Burgeon, A. Rao, J. Exp. Med. 184, 141 (1996)). pGFP-VIVIT carries the NFAT-specific inhibitory peptide VIVIT linked to GFP (H. Tran et al., Science 296, 530 (2002)). pHA-NFAT expresses NFATc1 tagged with the hemagglutinin peptide. pGFP-FoxO3a and pFoxO3a-A3 express, respectively, a GFP-FoxO3a fusion protein and a FoxO3a mutant in which the three Thr/Ser known to be phosphorylated by Akt and SGK are mutated into Ala, rendering FoxO3a insensitive to inhibition by Akt or IGF-1 (P. F. Dijkers et al., Mol. Cell. Biol. 20, 9138 (2000). M. Potente et al., J. Clin. Invest. 115, 2382 (2005)).

Transfection: For NFAT activity analysis, C2C12 myoblasts were transfected for 6 h with 0.5 μg of Gal4-Luc, 4 ng of the different pGal4-NFAT vectors along with 3 μg of pcDNA3-CnA, or empty vector, by using 10 μg of Lipofectamine 2000 (Invitrogen, Carlsbad, Calif.) according to the manufacturer's instructions. Where indicated, 1 μg of pGFP-VIVIT or the control vector pGFP was added. After 18 h in GM, cells were induced to differentiate in DM for 48 h and luciferase activity was measured. Where indicated, 10 ng/ml of cyclosporin A (or equivalent volume of EtOH) was added with DM. Jurkat T cells were grown and transfected as described (Luo, C., Burgeon, E. and Rao, A. Mechanisms of transactivation by nuclear factor of activated T cells-1. J. Exp. Med. 184, 141-147 (1996)), following the same protocols as for C2C12 cells. For FoxO activity analysis, C2C12 and HEK293 cells were transfected with 0.25 μg of p6xDBE-Luc and either 2.5 μg Cn expression vectors and 0.8 μg of pFoxO3a-A3 (or pcDNA3.1 as negative control), or 100 pmoles of CnAβ2 or CnAβ1 siRNA. Where indicated, 25 ng/ml IGF-1 was added to the culture for 16 h. IGF-1 and Cyclosporin a (CsA) were purchased from Sigma (St. Louis, Mo.). Stable C2C12 transfectants bearing the pcDNA3.1-CnAβ1 construct or the empty pcDNA3.1 vector were generated by selecting the cells with 0.5 mg/ml G418 after transfection. Two different polyclonal populations of transfectants were used in this study.

### Phosphoprotein Analysis

Quadriceps from uninjured or injured mice 12 days after cardiotoxin injection were lysed and examined for specific phosphorylated residues in different proteins by using the Kinetworks Phosphosite 4.1 western blot analysis (Kinexus, Vancouver, Canada). Two mice were used for each point and the results are expressed as the average fold induction over the value of the uninjured wild type mice for each phosphorylated site.

### Proliferation and Apoptosis Analysis

Cell proliferation was quantified using the MTT Cell Proliferation Assay (Sigma) 0 and 48 h after cell transfection. Nuclear fragmentation was determined in HEK293 cells 48 h after transfection with CnAβ1 or control siRNA by using the Cell Death Detection ELISA assay from Roche (Roche Molecular Biochemicals).

### Western Blots

Western blots were carried out as previously described (E. Lara-Pezzi et al., J. Biol. Chem. 279, 6553 (2004)). Antibodies against myogenin and p53 were obtained from Santa Cruz (Santa Cruz Biotechnology, Santa Cruz, Calif.) and used at 1 μg/ml. Anti-phosphoSer253-FoxO3a, anti-FoxO3a, anti-phospho-Rb and anti-phospho-ATM substrates (which recognize phosphorylated substrates of active ATM) and anti-Akt were purchased from Cell Signalling (Beverly, Mass.). Anti-Stag2 was a kind gift of Dr. José Luis Barbero (CNB, Madrid, Spain) (F. Mourkioti et al., J. Clin. Invest. In press. (2006)). Anti-HA was purchased from Roche. Secondary HRP-conjugated anti-rabbit and anti-mouse antibodies were obtained from Amersham (Amersham, UK) and used at a 1:3000 dilution.

To investigate the possible role of CnAβ1 in skeletal muscle regeneration, the inventors first studied the expression pattern of the two described CnAβ splicing variants in different adult muscles. Northern blot and quantitative RT-PCR analyses of CnAα, CnAβ2 and CnAβ1 mRNA levels showed equivalent expression levels of CnAα and Cd02 in all muscles analyzed, with weaker expression of CnAβ1 (FIGS. 1a and 1b). CnAβ1 and CdP2 expression showed differential regulation during C2C12 cell differentiation in culture with a progressive increase in CnAβ2 expression and a decline in CnAβ1 levels (FIG. 1c). In contrast during cardiotoxin-induced skeletal muscle regeneration the inventors found a progressive decrease of the CnAβ2 mRNA levels and a fast transient rise of CnAβ1 expression (FIG. 1d, 1e). These results suggest that while CnAβ2 may be involved in skeletal muscle development and maturation, CnAβ1 might play a role during muscle regeneration.

To further study the function of CnAβ1 in skeletal muscle, the inventors generated MLC-CnAβ1 and MLC-CnAα transgenic mice, expressing CnAβ1 and full length CnAα in a skeletal muscle-specific, post-mitotic restricted fashion (FIG. 6). The MLC-CnAα construct included the autoinhibitory domain also found in CnAβ2. The inventors first analyzed the transcript expression profile in the quadriceps muscles of wild type, MLC-CnAα and MLC-CnAβ1 mice at two months of age in the absence of any injury using oligonucleotide-based microarrays. The inventors found an induction of slow fiber-associated genes in MLC-CnAβ1 mice, but not in wild type and MLC-CnAα mice (FIG. 2a), including myosin light chains (Myl3, Myl2), troponins (Tnni1, Tnnt1), tropomyosins (Tpm3), and Serca2a. qRT-PCR analysis of slow and fast TnI mRNA expression in the different mice showed an up-regulation of TnI1 (slow) and slight decrease of TnI2 (fast) expression in MLC-CnAβ1 mice compared to wild type and MLC-CnAα (FIG. 2b), confirming the results obtained with the microarrays.

To determine whether CnAβ1 expression results in enhanced muscle regeneration, the inventors injected cardiotoxin into the tibialis anterior (TA) and quadriceps muscles of WT, MLC-CnAα, and MLC-CnAβ1 mice and allowed regeneration to proceed for 2, 6 or 12 days. Histological analysis of regenerating TA from the MLC-CnAβ1 mice 12 days after the injury showed lager myotubes with centralized nuclei and less extracellular matrix accumulation when compared to regenerating WT muscle (FIG. 3a). In contrast, MLC-CnAα mice showed increased matrix deposition, with small, separated myotubes and persistent presence of infiltrating inflammatory cells. The enhanced regenerative capacity shown by MLC-CnAβ1 mice was also reflected in the increased mean cross-sectional area (CSA) of their myotubes compared to those of WT (48% smaller mean CSA) and MLC-CnAα mice (57% smaller mean CSA).

To study the mechanisms responsible for the enhanced skeletal muscle regeneration observed in MLC-CnAβ1 mice, the inventors analyzed the transcription profile of the quadriceps from the uninjured and 12 day injured wild type, MLC-CnAα and MLC-CnAβ1 mice by using microarrays. A clear induction of genes involved in the immune response, extracellular matrix production, especially collagen, cell cycle and lysosome function was detected, paralleled by a strong decrease in the expression of genes associated with the mitochondria and energy production, likely reflecting myocyte necrosis. Although similar qualitative changes were observed in WT, MLC-CnAα and MLC-CnAβ1 mice during muscle regeneration, a strong decrease in the average expression of immune response-associated genes was observed in MLC-CnAβ1 mice 12 days after injection, compared to wild type and MLC-CnAα mice (FIG. 3c), indicating a faster resolution of the inflammation.

In particular, a clear reduction in the average expression of macrophage-associated genes was observed in MLC-CnAβ1 mice at this stage compared to the two other mouse lines. This decrease in the presence of macrophages was accompanied by a lower expression of extracellular matrix genes, mainly collagens (FIG. 3c), which are indicative of scarring. Thus, the improved muscle regeneration in MLC-CnAβ1 transgenic mice is characterized by decreased fibrosis and inflammation. MLC-CnAα mice, however, had impaired muscle regeneration and showed increased expression levels of genes associated with macrophage function and extracellular matrix production 12 days after injury (FIG. 3c), indicating enhanced inflammation and fibrosis. The increased collagen production observed in wild type and MLC-CnAα 12 days after injury was confirmed by qRT-PCR analysis (FIG. 3d) and was accompanied by an increase in the expression of the profibrotic cytokine TGF-β1.

Calcineurin exerts many of its actions through the activation of the transcription factor NF-AT. To find out whether CnAβ1 could behave as a constitutively active CnA, the inventors investigated the ability of the different calcineurin isoforms to activate the transcription factor NFAT both in vivo and in vitro. Western blot analysis showed additional dephosphorylation bands of NFATc3, NFATc1 and NFATc2 in nuclear extracts from the quadriceps of MLC-CnAβ1 mice (FIG. 4a), compared to wild type or MLC-CnAα mice (FIG. 4b). The inventors then transfected C2C12 cells with a reporter vector in which luciferase expression is controlled by three copies of the Gal4-responsive site, along with expression vectors for CnA isoforms and chimeras containing the Gal4 DNA-binding domain linked to different regions of the regulatory and transcription activation domains of NFAT. Whereas CnAα and CnAβ2 failed to activate MAT in the absence of other stimuli (FIG. 4c-4e), artificial truncation of their autoinhibitory domain (CnAα* and CnAβ*) resulted in activation of the Gal4-NFAT chimera bearing the transcription activation and regulatory domains (aa 1415). When the main phosphorylation sites in NFAT were removed (Gal4-NFAT-1-415Δ145-248 and Gal4-NFAT-1-171), activation of NFAT by the truncated forms was abolished (FIG. 4d-4e).

Interestingly, CnAβ1 was able to activate the three Gal4-NFAT forms tested, suggesting that the PxIxIT sequence of NFAT (aa 110-116), the major docking site for calcineurin, might be enough for its activation by CnAβ1 (FIG. 4c-4e). The inventors then cotransfected an expression vector encoding the NFAT-specific peptidic inhibitor VIVIT, which competes with the PxIxIT sequence in NFAT for calcineurin binding. VIVIT prevented the activation of NFAT-dependent transcription by CnAβ* and CnAβ1 (FIG. 4f), demonstrating the necessity of the calcineurin docking site for NFAT activation. When the inventors performed similar experiments in the presence of the immunosuppressive drug cyclosporine-A (CsA), which inhibits calcineurin by forming a complex with CnA and cyclophilin, activation of NFAT by CnAβ* was blocked. Interestingly, CsA failed to prevent NFAT activation by CnAβ1. These data are compatible with the constitutive nuclear localization of CnAβ1 previously observed and may reflect an interference of the NFAT rephosphorylation process in the nucleus, which would lead to increased NFAT dephosphorylation and activation.

To further unveil the signaling pathways underlying the effect of CnAβ1 on skeletal muscle regeneration, the inventors quantified the activation of different kinases by determining the phosphorylation state of specific residues within them by western blot. CnAβ1 expression in uninjured muscle resulted in ribosomal S6 kinase (RSK) activation and GSK-3a inhibition (FIG. 5a), suggesting a mechanism by which CnAβ1 might induce NFAT activation in a CsA-resistant manner. Interestingly, CnAβ1 expression led to activation of IKKα, but not IKKβ (FIG. 5a). Since loss of IKKEα activity, but not IKKβ, leads to increased inflammation, activation of IKK might explain the faster resolution of the inflammatory response and enhanced muscle regeneration observed in the MLC-CnAβ1 mice. On the contrary, MLC-CnAα mice showed activation of the MKK1/2 and MKK3/6 pathways, together with PKB and PKCδ phosphorylation. Twelve days after cardiotoxin injection, wild type and MLC-CnAα mice presented a strong activation of the PDK1/PKB/mTOR and the MKK1,2/ERK1,2 pathways, together with activation of PKCδ and PKCζ (FIG. 5b), all described to be involved in muscle growth. The active macrophage infiltration observed at this stage in WT and MLC-CnAα mice was supported by the activation of the leukocyte kinase Lyn (FIG. 5b) Interestingly, MLC-CnAβ1 mice presented no activation of Lyn 12 days after the injury and weaker activation of the muscle growth pathways (FIG. 3c), reflecting the increased muscle regeneration achieved by these mice already at this point.

### BIBLIOGRAPHY

1. Crabtree, G. R. and Olson, E. N. NFAT signaling: choreographing the social lives of cells. Cell 109, S67-S79 (2002).
2. Hogan, P., Chen, L., Nardone, J. and Rao, A. Transcriptional regulation by calcium, calcineurin, and NFAT. Genes Dev. 17, 2205-2232 (2003).
3. Guerini, D. and Klee, C. B. Cloning of human calcineurin A: evidence far two isozymes and identification of a polyproline structural domain. Proc. Natl. Acad. Sci. USA 86, 9183-9187 (1989).
4. Buckingham, M. Skeletal muscle formation in vertebrates. Curr. Opin. Genet. Dev. 11, 440-448 (2001).
5. McCullagh, K. J. et al. NFAT is a nerve activity sensor in skeletal muscle and controls activity-dependent myosin switching. Proc. Natl. Acad. Sci. USA 101, 10590-10595 (2004).
6. Horsley, V., Jansen, K. M., Mills, S. T. and Pavlath, G. K. IL-4 acts as a myoblast recruitment factor during mammalian muscle growth. Cell 113, 483-494 (2003).
7. Naya, F. J. et al. Stimulation of slow skeletal muscle fiber gene expression by calcineurin in vivo. J. Biol. Chem. 275, 4545-4548 (2000).
8. Talmadge, R. J. et al. Calcineurin activation influences muscle phenotype in a muscle specific fashion. BMC Cell Biol. 5, 28 (2004).
9. Serrano, A. L. et al. Calcineurin controls nerve activity-dependent specification of slow skeletal muscle fibers but not muscle growth. Proc. Natl. Acad. Sci. USA 98, 13108-13113 (2001).
10. Parsons, S. A., Wilkins, B. J., Bueno, O. F. and Molkentin, J. D. Altered skeletal muscle phenotypes in calcineurin Aalpha and Abeta gene-targeted mice. Mol. Cell. Biol. 23, 4331-4343 (2003).
11. St-Pierre S. J. et al. Glucocorticoid treatment alleviates dystrophic myofiber pathology by activation of the calcineurin/NF-AT pathway. FASEB J. 18, 1937-1939 (2004).
12. Dobrowolny, G. et al. Muscle expression of a local Igf-1 isoform protects motor neurons in an ALS mouse model. J; Cell Biol. 168, 193-199 (2005).
13. Musaro, A. et al. Localized Igf-1 transgene expression sustains hypertrophy and regeneration in senescent skeletal muscle. Nat. Gen. 27, 195-200 (2001).
14. Li, Y. et al. Transforming growth factor-beta 1 induces the differentiation of myogenic cells into fibrotic cells in injured skeletal muscle: a key event in muscle fibrogenesis. Am. J. Pathol. 164, 1007-1019 (2004).
15. Aramburu, J. et al. Affinity-driven peptide selection of an NFAT inhibitor more selective than cyclosporine A. Science 285, 2129-2133 (1999).
16. Zhu, J. and McKeoa, F. NF-AT activation requires suppression of Crm1-dependent export by calcineurin. Nature 398, 256-260 (1999).
17. Yang, T. T. C., Xiong, Q., Graef, I. A., Crabtree, G. R. and Chow, C. W. Recruitment of the Extracellular Signal-Regulated Kinase/Ribosomal S6 Kinase signaling pathway to the NFATc4 transcription activation complex. Mol. Cell. Biol. 25, 907-920 (2005).
18. Beals, C. R., Sheridan, C. M., Turck, C. W., Gardner, P. and Crabtree, G. R. Nuclear export of NF-ATc enhanced by glycogen synthase kinase-3. Science 275, 1931-1934 (1997).
19. Murphy, L. L. and Hughes, C. C. Endothelial cells stimulate T cell NFAT nuclear translocation in the presence of cyclosporin A: involvement of the wnt/glycogen synthase kinase-3 beta pathway. J. Immunol. 169, 3717-3725 (2002).
20. Lawrence, T., Bebien, M., Liu, G. Y., Nizet, V. and Karin, M. IKKalpha limits macrophage NF-kappaB activation and contributes to the resolution of inflammation. Nature 434, 1138-1143 (2005).
21. Good, J. A. L. et al. Protein kinase C isotypes controlled by phosphoinositide 3-kinase through the protein kinase PDK1. Science 281, 2042-2045 (1998).
22. Ohanna, M. et al. Atrophy of S6K1(−/−) skeletal muscle cells reveals distinct mTOR effectors for cell cycle and size control. Nat. Cell. Biol. 7, 286-294 (2005).
23. Pallafacchina, G., Calabria, E., Serrano, A. L., Kalhovde, J. M. and Schiaffino, S. A protein kinase B-dependent and rapamycin-sensitive pathway controls skeletal muscle growth but not fiber type specification. Proc. Natl. Acad. Sci. USA 99, 9213-9218 (2002).
24. Xu, Y., Harder, K. W., Huntington, N. D., ML, M. L. H. and Tarlinton, D. M. Lyn tyrosine kinase: accentuating the positive and the negative. Immunity 22, 9-18 (2005).
25. Musarb, A. et al. Stem cell-mediated muscle regeneration is enhanced by mIGF-1. Proc. Natl. Acad. Sci. USA 101, 1206-1210 (2004).
26. Luo, C., Burgeon, E. and Rao, A. Mechanisms of transactivation by nuclear factor of activated T cells-1. J. Exp. Med. 184, 141-147 (1996).

1. A method for treating skeletal muscle injury or degeneration or cancer in a subject, wherein said method comprises administering, to a subject in need of such treatment, an inhibitor or activator of the calcineurin subunit Aβ1 isoform (CnAβ1).

2. The method, according to claim 1, wherein the inhibitor or activator of CnAβ1 is selected from an inhibitor or activator of the biological activity of CnAβ1 and an inhibitor or activator of the expression of CnAβ1.

3. The method, according to claim 2, wherein the inhibitor or activator of the expression of CnAβ1 is selected from a vector encoding calcineurin CnAβ1, antisense oligonucleotides, siRNA and miRNA.

4. The method according to claim 3, wherein said vector is a viral vector.

5. The method according to claim 2, wherein the inhibitor or activator of the biological activity of CnAβ1 is selected from a vector encoding biologically inactive CnAβ1, an inhibitory peptide, a phosphatase inhibitor, and an inhibitor of the activity of a transcription factor of the FoxO family.

6. The method, according to claim 1, wherein the inhibitor or activator of CnAβ1 comprises cells that recombinantly express CnAβ1.

7. The method, according to claim 1, wherein the subject is a mammal.

8. The method according to claim 7, wherein the subject is a human.

9. The method, according to claim 1, wherein the treatment of skeletal muscle injury or degeneration or cancer comprises preventing or reversing skeletal or cardiac muscle atrophy, muscle wasting, enhancing skeletal muscle regeneration, decreasing scar formation in injured skeletal muscle, inducing myoblast differentiation, and/or blocking tumor cell growth.

10. The method, according to claim 1, wherein the method further comprises administering at least one additional pharmaceutically active ingredient that has an effect on calcineurin.

11. A method for screening a compound for the ability to inhibit or activate CnAβ1, comprising:
contacting a cell with a compound suspected to inhibit or activate CnAβ1;
assaying the contents of the cell to determine the amount and/or biological activity of CnAβ1; and
comparing the determined amount and/or biological activity of CnAβ1 to a predetermined level,
wherein a change of said amount and/or biological activity of CnAβ1 is indicative for a compound that inhibits or activates CnAβ1.

12. The method of claim 11, wherein the cell is a skeletal muscle cell or a tumor cell.

13. The method of claim 11, wherein the amount of calcineurin Aβ1 mRNA is determined.

14. A method for producing a pharmaceutical composition, comprising
a) identifying a compound that inhibits or activates CnAβ1 by contacting a cell with a compound suspected to inhibit or activate CnAβ1 by:
assaying the contents of the cell to determine the amount and/or biological activity of CnAβ1; and
comparing the determined amount and/or biological activity of CnAβ1 to a predetermined level,
wherein a change of said amount and/or biological activity of CnAβ1 is indicative for a compound that inhibits or activates CnAβ1; and
b) formulating the compound identified in part a) with a pharmaceutically acceptable carrier and/or excipient.

15. A pharmaceutical composition, produced according to claim 14.

16. The composition according to claim 15, wherein said composition is an antisense composition or a composition comprising cells that recombinantly express CnAβ1.

17. A method of modulating the expression of calcineurin Aβ1 in skeletal muscle tissue of a subject comprising administering a composition according to claim 15 to the subject such that the expression of calcineurin Aβ1 in the skeletal muscle tissue of the subject is modulated.

18. The method according to claim 1 which is used for enhancing skeletal muscle regeneration in a subject by expressing or overexpressing calcineurin Aβ1 in or proximate to skeletal, muscle tissue in the subject.

19. The method according to claim 1, which is used for treating skeletal muscle injury in a subject by expressing or overexpressing calcineurin Aβ1 in or proximate to skeletal, muscle tissue in the subject.

20. The method according to claim 1, which is used for treating skeletal muscle degeneration in a subject by expressing or overexpressing calcineurin Aβ1 in or proximate to degenerated or degenerating skeletal muscle tissue in the subject.

21. The method according to claim 1 which is used for treating cancer in a subject, by administering to said subject an effective amount of an inhibitor of the expression and/or biological activity of CnAβ1.

22. The method according to claim 1, which is used for inducing cellular differentiation by inhibiting the expression of CnAβ1 in myoblasts and/or tumor cells.

23. The method according to claim 1, wherein expressing or overexpressing calcineurin Aβ1 comprises delivering cells that express calcineurin Aβ1 to said subject.
